SEQUENCE LISTING (154 sequences)

SEQ ID NO: 1 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 2 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 3 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 4 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 5 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 6 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGVKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 7 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLNKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 8 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 9 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 10 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGDKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 11 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLLKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 12 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 13 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 14 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 15 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 16 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 17 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 18 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 19 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 20 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 21 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 22 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 23 - protein, 1370 aa, synthetic construct; identical to SEQ ID NO: 1

SEQ ID NO: 24 - protein, 1370 aa, synthetic construct; identical to SEQ ID NO: 1

SEQ ID NO: 25 - protein, 1370 aa, synthetic construct; identical to SEQ ID NO: 1

SEQ ID NO: 26 - protein, 1370 aa, synthetic construct; identical to SEQ ID NO: 1

SEQ ID NO: 27 - protein, 1370 aa, synthetic construct; identical to SEQ ID NO: 1

SEQ ID NO: 28 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDPKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 29 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDPKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 30 - protein, 1370 aa, synthetic construct
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDPKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKL

SEQ ID NO: 31 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 32 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 33 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 34 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 35 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtacttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 36 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 37 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 38 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 39 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 40 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 41 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 42 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 43 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 44 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 45 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 46 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 47 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 48 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 49 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 50 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 51 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 52 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 53 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 54 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 55 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 56 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 57 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 58 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacccgaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 59 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgaccctaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 60 - DNA, 4110 bp, synthetic construct
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgaccctaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctg

SEQ ID NO: 61 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 62 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 63 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 64 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 65 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGVKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 66 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLNKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 67 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 68 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 69 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGDKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 70 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLLKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 71 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 72 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 73 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 74 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 75 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 76 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 77 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 78 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 79 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 80 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 81 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 82 - protein, 1410 aa, synthetic construct; identical to SEQ ID NO: 77

SEQ ID NO: 83 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 84 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 85 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 86 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 87 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILEALLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQRLNSYNDFKAIVGDKVDSDDYRDKIEEIIKLIVLYGDDKSYLQKKIKAGYGKYFTDSEIKKMAGLNYKDWGRLSKKLLTGLEGALKITGERGSIIHFMREYNLNLMELMSASFTFTEEIQKLNPVDDRKLSYEMVDELYLSPSVKRMLWQSLRIVDEIKNIMGTDPKKIFIEMARGKEEVKARKESRKNQLLKFYKDGKKAFISEIGEERYSYLLSEIEGEEFNKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLLRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 88 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDPKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 89 - protein, 1410 aa, synthetic construct
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEYFDDSAIKSVDFSTTSFDDKYSDYELALGVKIALVNILKEIYDSSILENLNKEADKSKDGNKYISNAFVKKYNKHGQDLKEFKRLVRQYHKSAYFDIFRSEKVNDNYVSYTKSSISSNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDPKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGAFFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSRKTNNIGNYQEFKIINQSITGLFENEVDLLKLGSYPYDVPDYAAAPKKKRKVAASHHHHHHHH

SEQ ID NO: 90 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 91 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 92 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 93 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtacttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 94 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 95 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 96 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 97 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 98 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 99 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 100 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 101 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 102 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 103 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 104 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 105 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 106 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 107 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 108 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 109 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 110 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 111 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 112 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 113 - DNA, 4232 bp, synthetic construct
  source 1..4232: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcatta

SEQ ID NO: 114 - DNA, 4232 bp, synthetic construct
  source 1..4232: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcatta

SEQ ID NO: 115 - DNA, 4232 bp, synthetic construct
  source 1..4232: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcatta

SEQ ID NO: 116 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgaggcgctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagaggttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcattgaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacccgaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaatttaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgcttcggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 117 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgaccctaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 118 - DNA, 4233 bp, synthetic construct
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgagtatttcgacgactctgcgataaagtccgttgacttctcaactacctctttcgacgacaagtactctgactatgaactcgctctgggtgttaagatcgctctggtcaacatccttaaggaaatttacgatagctccatcctcgagaacctgaataaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcagtaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgaccctaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgtttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagtaggaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 119 - DNA, 25 bp, synthetic construct
  source 1..25: mol_type = other DNA
cagctgcgggaaagggattcccagg

SEQ ID NO: 120 - DNA, 25 bp, synthetic construct
  source 1..25: mol_type = other DNA
cagctgcgggaatgggattcccagg

SEQ ID NO: 121 - DNA, 25 bp, synthetic construct
  source 1..25: mol_type = other DNA
cagctgagggaaaggaattcccagg

SEQ ID NO: 122 - DNA, 25 bp, synthetic construct
  source 1..25: mol_type = other DNA
gtgtcaagccccagaggccacaggg

SEQ ID NO: 123 - DNA, 25 bp, synthetic construct
  source 1..25: mol_type = other DNA
gtgtcaagccccagaggacacaggg

SEQ ID NO: 124 - DNA, 25 bp, synthetic construct
  source 1..25: mol_type = other DNA
aagccaaaccccaaaggccacacgg

SEQ ID NO: 125 - DNA, 25 bp, synthetic construct
  source 1..25: mol_type = other DNA
tcacagcgggtgtagactccgaggg

SEQ ID NO: 126 - DNA, 25 bp, synthetic construct
  source 1..25: mol_type = other DNA
gcagtccgggctgggagcgggtggg

SEQ ID NO: 127 - DNA, 25 bp, synthetic construct
  source 1..25: mol_type = other DNA
acagtcctggctgggagcaggtggg

SEQ ID NO: 128 - DNA, 25 bp, synthetic construct
  source 1..25: mol_type = other DNA
tgctcagacacgcggtgcagcaggg

SEQ ID NO: 129 - DNA, 25 bp, synthetic construct
  source 1..25: mol_type = other DNA
gtttgccaccagtacagggcagcgg

SEQ ID NO: 130 - DNA, 25 bp, synthetic construct
  source 1..25: mol_type = other DNA
agaagtcctaatggtagcacctggg

SEQ ID NO: 131 - RNA, 22 nt, synthetic construct
  source 1..22: mol_type = other RNA
cagctgcgggaaagggattccc

SEQ ID NO: 132 - RNA, 22 nt, synthetic construct
  source 1..22: mol_type = other RNA
cagctgcgggaatgggattccc

SEQ ID NO: 133 - RNA, 22 nt, synthetic construct
  source 1..22: mol_type = other RNA
gtgtcaagccccagaggccaca

SEQ ID NO: 134 - RNA, 22 nt, synthetic construct
  source 1..22: mol_type = other RNA
gtgtcaagccccagaggacaca

SEQ ID NO: 135 - RNA, 22 nt, synthetic construct
  source 1..22: mol_type = other RNA
tcacagcgggtgtagactccga

SEQ ID NO: 136 - RNA, 22 nt, synthetic construct
  source 1..22: mol_type = other RNA
gcagtccgggctgggagcgggt

SEQ ID NO: 137 - RNA, 22 nt, synthetic construct
  source 1..22: mol_type = other RNA
tgctcagacacgcggtgcagca

SEQ ID NO: 138 - RNA, 22 nt, synthetic construct
  source 1..22: mol_type = other RNA
gtttgccaccagtacagggcag

SEQ ID NO: 139 - RNA, 22 nt, synthetic construct
  source 1..22: mol_type = other RNA
agaagtcctaatggtagcacct

SEQ ID NO: 140 - RNA, 80 nt, synthetic construct
  source 1..80: mol_type = other RNA
gtttgagagttatgaaaatgacgagttcaaataaaaatttattcaaaccgcctatttataggccgcagatgttctgcttt

SEQ ID NO: 141 - RNA, 80 nt, synthetic construct; identical to SEQ ID NO: 140
  source 1..80: mol_type = other RNA

SEQ ID NO: 142 - RNA, 102 nt, synthetic construct
  source 1..102: mol_type = other RNA
ttagagtctctcagctggtacagtttgagagttatgaaaatgacgagttcaaataaaaatttattcaaaccgcctatttataggccgcagatgttctgcttt

SEQ ID NO: 143 - DNA, 25 bp, synthetic construct
  source 1..25: mol_type = other DNA
agaagtcctaatggtagcacctggg

SEQ ID NO: 144 - DNA, 25 bp, synthetic construct
  source 1..25: mol_type = other DNA
cctgtactggtggcaaacccagtgg

SEQ ID NO: 145 - DNA, 25 bp, synthetic construct
  source 1..25: mol_type = other DNA
gcagtacaggtggcaaacccagagg

SEQ ID NO: 146 - DNA, 25 bp, synthetic construct
  source 1..25: mol_type = other DNA
gtgctgcagaagggattccatgagg

SEQ ID NO: 147 - DNA, 25 bp, synthetic construct
  source 1..25: mol_type = other DNA
atgctgcagaagggattccaagggg

SEQ ID NO: 148 - DNA, 25 bp, synthetic construct
  source 1..25: mol_type = other DNA
tgggtgagtgagtgtgtgcgtgtgg

SEQ ID NO: 149 - RNA, 22 nt, synthetic construct
  source 1..22: mol_type = other RNA
agaagtcctaatggtagcacct

SEQ ID NO: 150 - RNA, 22 nt, synthetic construct
  source 1..22: mol_type = other RNA
cctgtactggtggcaaacccag

SEQ ID NO: 151 - RNA, 22 nt, synthetic construct
  source 1..22: mol_type = other RNA
gcagtacaggtggcaaacccag

SEQ ID NO: 152 - RNA, 22 nt, synthetic construct
  source 1..22: mol_type = other RNA
gtgctgcagaagggattccatg

SEQ ID NO: 153 - RNA, 22 nt, synthetic construct
  source 1..22: mol_type = other RNA
atgctgcagaagggattccaag

SEQ ID NO: 154 - RNA, 22 nt, synthetic construct
  source 1..22: mol_type = other RNA
tgggtgagtgagtgtgtgcgtg

(19) United States
(12) Patent Application Publication
(10) Pub. No.: US 2026/0226439 A1
(43) Pub. Date: Aug. 6, 2026

(54) Engineered Omni-50 Nuclease Variants

(71) Applicant: EmendoBio Inc., Wilmington, DE (US)

(72) Inventors: Lior IZHAR, Tel Aviv (IL); Liat ROCKAH, Rishon LeZion (IL); Milit MAROM DAVID, Tel Aviv (IL); Shira WARSZAWSKI, Rehovot (IL)

(21) Appl. No.: 19/152,173

(22) PCT Filed: Feb. 8, 2024
(86) PCT No.: PCT/US2024/015015

§ 371 Date: Jul. 30, 2025

Related U.S. Application Data
(60) Provisional application No. 63/483,851, filed on Feb. 8, 2023.
(60) Provisional application No. 63/491,380, filed on Mar. 21, 2023.
(60) Provisional application No. 63/511,795, filed on Jul. 3, 2023.
(60) Provisional application No. 63/511,927, filed on Jul. 5, 2023.

(51) Int. Cl. C12N 9/22; C12N 5/071; C12N 15/10; C12N 15/113; C12N 15/90

(52) U.S. Cl. CPC C12N 9/222; C12N 5/0602; C12N 15/102; C12N 15/113; C12N 15/907; C12N 2310/20; C12N 2510/00

(57) ABSTRACT

The present invention provides a composition comprising a non-naturally nuclease variant having at least 90% identity to SEQ ID NO: 1 and comprising an amino acid substitution in at least one of the following positions: N300, G614, N698, E836, T939, L1100, D252, D281, L302, N368, S1339, and S779 relative to SEQ ID NO: 1.

This application claims the benefit of: (i) U.S. Provisional Application No. 63/483,851, filed Feb. 8, 2023; (ii) U.S. Provisional Application No. 63/491,380, filed Mar. 21, 2023; (iii) U.S. Provisional Application No. 63/511,795, filed Jul. 3, 2023; and (iv) U.S. Provisional Application No. 63/511,927, filed Jul. 5, 2023, the contents of each of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “102322_040393_PCT_Eng_Omni_50_Variants.xml”, which is 462,633 bytes in size, and which was created on Jul. 5, 2023, in the IBM-PC machine format, having an operating system compatibility with MS-Windows, which is contained in the XML file filed Feb. 8, 2024, as part of this application.

### FIELD OF THE INVENTION

The present invention is directed to, inter alia, composition and methods for genome editing.

### BACKGROUND OF THE INVENTION

The Clustered Regularly Interspaced Short Palindromic Repeat (CRISPR) systems of bacterial and archaeal adaptive immunity show extreme diversity of protein composition and genomic loci architecture. The CRISPR systems have become important tools for research and genome engineering. Nevertheless, many details of CRISPR systems have not been determined and the applicability of CRISPR nucleases may be limited by sequence specificity requirements, expression, or delivery challenges. Different CRISPR nucleases have diverse characteristics such as: size, PAM site, on target activity, specificity, cleavage pattern (e.g. blunt, staggered ends), and prominent pattern of indel formation following cleavage. Different sets of characteristics may be useful for different applications. For example, some CRISPR nucleases may be able to target particular genomic loci that other CRISPR nucleases cannot due to limitations of the PAM site. In addition, some CRISPR nucleases currently in use exhibit pre-immunity, which may limit in vivo applicability. See Charlesworth et al., Nature Medicine (2019) and Wagner et al., Nature Medicine (2019). Accordingly, discovery, engineering, and improvement of novel CRISPR nucleases and the RNA molecules that activate and target them is of importance.

### SUMMARY OF THE INVENTION

Disclosed herein are engineered Clustered Regularly Interspaced Short Palindromic Repeats (CRISPRs)/CRISPR-associated OMNI-50 nucleases with improved activity and their use in genomic engineering, epigenomic engineering, genome targeting, genome editing, and in vitro diagnostics.

According to some aspects of the invention, there is provided a variant of an OMNI-50 nuclease with improved activity and/or specificity as compared to the wild-type OMNI-50 nuclease, as well as methods of using the improved variants. Advantageously, when the engineered variant OMNI-50 nucleases are active in a CRISPR endonuclease system the CRISPR endonuclease system displays increased on-target editing activity relative to a wild-type CRISPR endonuclease system in which a wild-type OMNI-50 nuclease is active. For example, an engineered variant OMNI-50 nuclease may display improved nuclease activity at a target region which contains a heterozygous SNP present in only the targeted allele and not present in the non-targeted allele. In some embodiments, the engineered variant OMNI-50 nuclease has reduced off-target effects.

According to some embodiments of the present invention, there is provided a variant of OMNI-50 nuclease protein comprising a sequence that is at least 80% identical to the amino acid sequence of wild-type OMNI-50 nuclease protein (SEQ ID NO: 1).

In some embodiments, the invention provides a composition comprising a non-naturally occurring OMNI-50 nuclease variant having at least 80%, 85%, 90%, 95%, or 97% identity to wild-type OMNI-50 protein sequence (SEQ ID NO: 1) comprising an amino acid substitution in at least 1, 2, 3, 4, 5, 6, 7, 8, 9, 10, 11, or 12 of the following positions: N300, G614, N698, E836, T939, L1100, D252, D281, L302, N368, S1339, and S779 compared to SEQ ID NO: 1. Each possibility represents a separate embodiment of the present disclosure, and can be combined at will.

In some embodiments, the invention provides a composition comprising a non-naturally occurring OMNI-50 nuclease variant having at least 80%, 85%, 90%, 95%, or 97% identity to wild-type OMNI-50 protein sequence (SEQ ID NO: 1) comprising an amino acid substitution in at least 1, 2, 3, 4, 5, or 6 of the following positions: N300, G614, N698, E836, T939, and L1100 compared to SEQ ID NO: 1. Each possibility represents a separate embodiment of the present disclosure, and can be combined at will.

In some embodiments, the invention provides a composition comprising a non-naturally occurring OMNI-50 nuclease variant having at least 80%, 85%, 90%, 95%, or 97% identity to wild-type OMNI-50 protein sequence (SEQ ID NO: 1) comprising an amino acid substitution in at least 1, 2, 3, 4, 5, or 6 of the following positions: D252, D281, L302, N368, L1100, and S1339, compared to SEQ ID NO: 1. Each possibility represents a separate embodiment of the present disclosure and can be combined at will in any combination.

In some embodiments, the invention provides a composition comprising a non-naturally occurring OMNI-50 nuclease variant having at least 80%, 85%, 90%, 95%, or 97% identity to wild-type OMNI-50 protein sequence (SEQ ID NO: 1) comprising an amino acid substitution in at least 1, 2, 3, 4, 5, 6, 7, or 8 of the following positions: N300, G614, N698, S779, E836, T939, L1100, and S1339, compared to SEQ ID NO: 1. Each possibility represents a separate embodiment of the present disclosure and can be combined at will in any combination.

In some embodiments, the non-naturally occurring OMNI-50 nuclease variant exhibits improved activity fidelity and/or targeting, compared to wild-type OMNI-50 protein (SEQ ID NO: 1). In some embodiments, such variant comprises an amino acid substitution at position L1100 compared to SEQ ID NO: 1. In some embodiments, such variant further comprises a substitution in at least 1, 2, 3, 4, or 5 of the following positions: N300, G614, N698, E836, T939, compared to SEQ ID NO: 1. In some embodiments, such variant further comprises substitution in at least 1, 2, 3, 4, or 5 of the following positions: D252, D281, L302, N368, and S1339, compared to SEQ ID NO: 1. In some embodiments, such variant further comprises a substitution at position S779, compared to SEQ ID NO: 1.

In some embodiments, the non-naturally occurring OMNI-50 nuclease variant exhibits increased specificity, compared to wild-type OMNI-50 protein (SEQ ID NO: 1). In some embodiments, such variant comprises an amino acid substitution at position S1339 compared to SEQ ID NO: 1. In some embodiments, such variant further comprises a substitution in at least 1, 2, 3, 4, 5, or 6 of the following positions: N300, G614, N698, E836, T939, and L1100 compared to SEQ ID NO: 1. In some embodiments, such variant further comprises substitution in at least 1, 2, 3, or 4 of the following positions: D252, D281, L302, and N368, compared to SEQ ID NO: 1. In some embodiments, such variant further comprises a substitution at position S779, compared to SEQ ID NO: 1.

In some embodiments, the non-naturally occurring OMNI-50 nuclease variant exhibits increased stability compared to wild-type OMNI-50 protein (SEQ ID NO: 1). In some embodiments, such variant comprises an amino acid substitution at position S779 compared to SEQ ID NO: 1.

In some embodiments, the invention provides a non-naturally occurring OMNI-50 nuclease variant having a sequence of at least 80%, 85%, 90%, 95%, or 97% identity to wild-type OMNI-50 protein sequence (SEQ ID NO: 1) comprising at least 1, 2, 3, 4, 5, 6, 7, 8, 9, 10, 11, or 12 of the following amino acid substitutions: N300A, G614R, N698L, E836F, T939L, L1100F, D252Y, D281V, L302N, N368S, S1339R, and S779P, compared to SEQ ID NO: 1. Each possibility represents a separate embodiment of the present disclosure and can be combined at will in any combination.

In some embodiments, the invention provides a non-naturally occurring OMNI-50 nuclease variant having a sequence of at least 80%, 85%, 90%, 95%, or 97% identity to wild-type OMNI-50 protein sequence (SEQ ID NO: 1) comprising at least 1, 2, 3, 4, 5, or 6 of the following amino acid substitutions: N300A, G614R, N698L, E836F, T939L, and L1100F, compared to SEQ ID NO: 1. Each possibility represents a separate embodiment of the present disclosure and can be combined at will in any combination.

In some embodiments, the invention provides a non-naturally occurring OMNI-50 nuclease variant having a sequence of at least 80%, 85%, 90%, 95%, or 97% identity to wild-type OMNI-50 protein sequence (SEQ ID NO: 1) comprising at least 1, 2, 3, 4, 5, or 6 of the following amino acid substitutions: D252Y, D281V, L302N, N368S, L1100F, and S1339R, compared to SEQ ID NO: 1. Each possibility represents a separate embodiment of the present disclosure and can be combined at will in any combination.

In some embodiments, the invention provides a non-naturally occurring OMNI-50 nuclease variant having a sequence of at least 80%, 85%, 90%, 95%, or 97% identity to wild-type OMNI-50 protein sequence (SEQ ID NO: 1) comprising at least 1, 2, 3, 4, 5, 6, 7, or 8 of the following amino acid substitutions: N300A, G614R, N698L, S779P, E836F, T939L, L1100F, and S1339R, compared to SEQ ID NO: 1. Each possibility represents a separate embodiment of the present disclosure and can be combined at will in any combination.

According to embodiments of the present invention, there is provided a composition comprising a non-naturally nuclease variant having at least 90% identity to SEQ ID NO: 1 and comprising an amino acid substitution in at least one of the following positions: N300, G614, N698, E836, T939, L1100, D252, D281, L302, N368, S1339, and S779 relative to SEQ ID NO: 1.

### BRIEF DESCRIPTION OF THE DRAWINGS

FIGS. 1A-1D. V6552 and V6172 show increased activity and specificity in a homozygous cell line for the ELANE_g58Ref target site. Proteins were tested in homozygous HSC cells with ELANE g58Ref. Percent (%) total edits at the target and off-target sites were calculated by next-generation sequencing (NGS) as indel frequency out of the total aligned reads. (A) On-target for g58Ref V6552 vs. WT OMNI-50. (B) On-target for g58Ref V6172 vs. WT OMNI-50. (C) Off-target for g58Ref V6552 vs. WT OMNI-50. (D) Off-target for g58Ref V6172 vs. WT OMNI-50.

FIGS. 2A-2C. V6552 and V6172 show broad activity on different targets. (A) WT OMNI-50 and V6552 were tested in Jurkat cells on different targets (Table 5) and the % total edits was calculated by NGS as indel frequency out of the total aligned reads. (B) WT OMNI-50 and V6552 were tested in HSCs on ELANE g38 (Table 5) and the % total edits was calculated by NGS as indel frequency out of the total aligned reads. (C) WT OMNI-50 and V6172 were tested in Jurkat cells on ELANE_g38 (Table 5) and the % total edits was calculated by NGS as indel frequency out of the total aligned reads.

FIGS. 3A-3E. V6552 and V6172 show increase discriminations in heterozygous cell line for the selected target site (ELANE_g58Ref). Proteins were tested in HSC heterozygous cells with ELANE g58Ref or g58Alt. Percent (%) total edits at the target and off-target sites were calculated by NGS as indel frequency out of the total aligned reads. Discrimination is determined by reduction in the fraction of the non-edited targeted allele versus the non-targeted allele. (A) Unedited fractions of each allele (Alt or Ref), and on-target for g58Ref or g58Alt with WT OMNI-50 and V6552. (B) Off-target for g58Ref with V6552 vs. WT OMNI-50. (C) Unedited fractions of each allele (Alt or Ref), and on-target for g58Ref or g58Alt (D) of WT OMNI-50 and V6172. (E) Off-target for g58Ref with V6172 vs. WT OMNI-50. The V6172 optimized nuclease variant is both active and discriminatory, showing a reduction of the alternative allele while the reference allele is kept intact.

FIGS. 4A-4D. V6172 and V6552 also show increased discrimination at other target sites in a heterozygous cell line. Proteins were tested in heterozygous HSC cells with ELANE_g62Ref. Percent (%) total edits at the target and off-target sites were calculated by NGS as indel frequency out of the total aligned reads. (A) On-target for g62Ref with WT OMNI-50 and V6552. (B) Off-target for g62Ref with V6552 vs. WT OMNI-50. (C) On-target for g62Ref with WT OMNI-50 and V6172. (D) Off-target for g62Ref with V6172 vs. WT OMNI-50.

FIGS. 5A-5B. V6172 has higher fidelity than WT OMINI-50 at another tested site. Proteins were tested in heterozygous HSC cells with ELANE_g35. Percent (%) total edits at the target and off-target sites were calculated by NGS as indel frequency out of the total aligned reads. (A) On-target for ELANE g35 with WT OMNI-50 and V6172. (B) Off-target for ELANE_g35 with V6172 vs. WT OMNI-50.

FIGS. 6A-6B. V6552 single mutations that contribute to an increase in activity. Variants containing only a single mutation present in V6552 were tested in homozygous HSC cells with ELANE_g58Ref, and editing levels were calculated for the on-target (A) and off-target (B) sites using NGS as indel frequency out of the total aligned reads. Mutations L1100F (V7896) and S1339R (V7101) display a major contribution to activity and the variant containing both of these mutations (V7492) has similar activity to V6552. S1339R contributes to specificity as demonstrated by the lower off-target edits of V7101.

FIGS. 7A-7B. Removing a single mutation from V6552 and its contribution to activity and specificity. Variants containing five out of the six mutations present in V6552 were tested in homozygous ELANE g58Ref HSC cells and editing levels were calculated for the on-target (A) and off-target (B) sites using NGS as indel frequency out of the total aligned reads. Removing L1100F or S1339R (V7257 and V7256, respectively) reduced the activity relative to V6552, highlighting the importance of those mutations to activity. D252Y and L302N contribute to specificity as demonstrated by the increase off-target edits (B) when they are removed (V7253 and V7255, respectively).

FIGS. 8A-8B. V6172 single mutations that contribute to an increase in activity and fidelity. Variants containing only a single mutation present in V6172 were tested in homozygous HSC cells to ELANE_g58Ref and editing levels were calculated for the on-target (A) and off-target (B) sites using NGS as indel frequency out of the total aligned reads. Mutation L1100F appears to contribute to activity the most. The other mutations provide additional increases to activity. The single mutants of N300A (V7239), N698L (V7241), and T939L (V7243) show a reduction of off-target levels, indicating their role in fidelity (FIG. 8B).

FIGS. 9A-9B. Removing a single mutation from V6172 and its contributions to activity and specificity. Variants containing five out of the six mutations from V6172 were tested in homozygous ELANE_g58Ref HSC cells and editing levels were calculated for the on-target (A) and off-target (B) sites using NGS as indel frequency out of the total aligned reads. Removing L1100F (V7143) reduced the activity relative to V6172, highlighting the importance of this mutation to activity. When removing only one of the mutations, it was shown that N300A (V7138), N698L (V7140), and T939L (V7142) increases off-target edits compared to V6172 (B), showing the contributions of these mutations to fidelity.

FIG. 10. Removing a single mutation from V6172 and its contributions to discrimination. Variants containing five out of the six mutations from V6172 were tested in heterozygous ELANE_g58Alt HSC cells and editing levels were calculated for the on-target site using NGS as indel frequency out of the total aligned reads. Discrimination is determined by reduction in the fraction of the non-edited targeted allele versus the non-targeted allele. Unedited fractions of each allele (Alt or Ref), and on-target edits of g58Alt for each variant are shown. When removing only one of the mutations, it was shown that N300A (V7138), N698L (V7140), and T939L (V7142) reduce discrimination compared to V6172 as shown by a reduction in the non-edited fraction of the non-targeted allele (Ref). These data show that the mutations also contribute to nuclease discrimination.

FIG. 11. Mutation S779P increases thermal stability of OMNI-50. A S779P mutation was introduced to WT OMNI-50 (forming variant V7261) and to variant V6552 (forming variant V7281). The thermal stability of each protein was tested by incubating the proteins at 25° C. and 44° C. and measuring the percent residual activity, as indicated by DNA cleavage, at the elevated temperature.

FIGS. 12A-12B. Variants V6552, V6172, and V7765 show increased activity in a cell line homozygous for RPE65 and VEGF_A3 targets. Proteins were tested in homozygous HSC cells with RPE65_g13 and VEGF_A3_g10 targets. Percent (%) total edits at the target and off-target sites were calculated by next-generation sequencing (NGS) as indel frequency out of the total aligned reads. (A) On-target for RPE65 with V6552, V6172, V7765 vs. WT OMNI-50. (B) On-target for VEGF3 with V6552, V6172, V7765 vs. WT OMNI-50.

FIGS. 13A-13I. Variants V6552, V6172, and V7765 show increased activity and specificity in a cell line homozygous for the other target sites (ELANE_g62Ref, ELANE_g58Ref SARM1, and FANCF). Proteins were tested in HSC cells homozygous for ELANE_g62Ref, ELANE_g58Ref, and in SH-SY5Y cells with SARML. Percent (%) total edits at the target and off-target sites were calculated by next-generation sequencing (NGS) as indel frequency out of the total aligned reads. (A) On-target for ELANE_g62Ref with V6552, V6172, V7765 vs. WT OMNI-50. (B) Off-target1 forELANE_g62Refwith V6552, V6172, V7765 vs. WT OMNI-50. (C) Off-target2 for ELANE g62Ref with V6552, V6172, V7765 vs. WT OMNI-50. (D) On-target for ELANE_g58Ref with V6552, V6172, V7765 vs. WT OMNI-50. (E) Off-target for ELANE g58Ref with V6552, V6172, V7765 vs. WT OMNI-50. (F) On-target for SARM1 with V6552, V6172, V7765 vs. WT OMNI-50. (G) Off-target for SARM1 with V6552, V6172, V7765 vs. WT OMNI-50. (H) On-target for FANCF with V6552, V6172, V7765 vs. WT. (I) Off-target for FANCF with V6552, V6172, V7765 vs. WT OMNI-50.

### DETAILED DESCRIPTION OF THE INVENTION

Disclosed herein are engineered Clustered Regularly Interspaced Short Palindromic Repeats (CRISPRs)/CRISPR-associated OMNI-50 nuclease variants with altered and improved characteristics and their use in genomic engineering, epigenomic engineering, genome targeting, genome editing, and in vitro diagnostics.

According to embodiments of the present invention, there is provided a composition comprising a non-naturally nuclease variant having at least 90% identity to SEQ ID NO: 1 and comprising an amino acid substitution in at least one of the following positions: N300, G614, N698, E836, T939, L1100, D252, D281, L302, N368, S1339, and S779 relative to SEQ ID NO: 1. In some embodiments, the nuclease variant comprises an amino acid substitution in at least one of the following positions: L1100, S1339, and/or S779.

In some embodiments, the nuclease variant comprises an amino acid substitution at L1100.

In some embodiments, the nuclease variant comprises an amino acid substitution at S1339.

In some embodiments, the nuclease variant comprises an amino acid substitution at positions L1100 and S1339.

In some embodiments, the nuclease variant comprises an amino acid substitution at S779.

In some embodiments, the nuclease variant comprises an amino acid substitution at each of positions L1100, S1339, and S779.

In some embodiments, the nuclease variant comprises an amino acid substitution at position L1100 and the amino acid substituted for leucine is a histidine (H), phenylalanine (F), tryptophan (W), or tyrosine (Y).

In some embodiments, the nuclease variant comprises an amino acid substitution at position L1100 and the amino acid substituted for leucine is a phenylalanine (L1100F).

In some embodiments, the nuclease variant comprises an amino acid substitution at position S1339 and the amino acid substituted for serine is an arginine (R), lysine (K), or histidine (H).

In some embodiments, the nuclease variant comprises an amino acid substitution at position S1339 and the amino acid substituted for serine is an arginine (S1339R).

In some embodiments, the nuclease variant comprises an amino acid substitution at position S779 and the amino acid substituted for serine is a glycine (G), alanine (A), valine (V), cysteine (C), proline (P), leucine (L), isoleucine (I), methionine (M), tryptophan (W), phenylalanine (F), aspartic acid (D), asparagine (N), or histidine (H).

In some embodiments, the nuclease variant comprises an amino acid substitution at position S779 and the amino acid substituted for serine is a proline (S779P).

In some embodiments, the nuclease variant comprises an amino acid substitution in at least one of the following positions: N300, G614, N698, E836, T939, and L1100.

In some embodiments, the nuclease variant comprises at least one of the following amino acid substitutions: N300A, G614R, N698L, E836F, T939L, and L1100F.

In some embodiments, the nuclease variant comprises an amino acid substitution in at least one of the following positions: D252, D281, L302, N368, L1100, and S1339.

In some embodiments, the nuclease variant comprises at least one of the following amino acid substitutions: D252Y, D281V, L302N, N368S, L1100F, and S1339R.

In some embodiments, the nuclease variant comprises an amino acid substitution in at least one of the following positions: N300, G614, N698, S779, E836, T939, L1100, and S1339.

In some embodiments, the nuclease variant comprises at least one of the following amino acid substitutions: N300A, G614R, N698L, S779P, E836F, T939L, L1100F, and S1339R.

In some embodiments, the nuclease variant has a wild-type OMNI-50 protein sequence (SEQ ID NO: 1) comprising an amino acid substitution in at least one of the following positions: N300, G614, N698, E836, T939, L1100, D252, D281, L302, N368, S1339, and S779.

In some embodiments, the nuclease variant comprises at least one of the following amino acid substitutions: N300A, G614R, N698L, E836F, T939L, L1100F, D252Y, D281V, L302N, N368S, S1339R, and S779P.

In some embodiments, the nuclease variant comprises an amino acid sequence of any one of SEQ ID NOs: 2-30.

In some embodiments, the nuclease variant further comprises at least one nuclear localization sequence (NLS).

In some embodiments, the nuclease variant further comprises at least one affinity tag.

In some embodiments, the nuclease variant is linked to another protein to form a fusion protein.

In some embodiments, the nuclease variant is a nickase or is catalytically inactive.

According to embodiments of the present invention, there is also provided a composition comprising a polynucleotide encoding the any one of the nuclease variants described herein, preferably wherein the polynucleotide is a DNA or RNA molecule, preferably an mRNA molecule.

In some embodiments, the composition further comprises a single-guide RNA (sgRNA) molecule, crRNA molecule, and/or a tracrRNA molecule, or a DNA molecule encoding a single-guide RNA (sgRNA) molecule, crRNA molecule, and/or a tracrRNA molecule.

According to embodiments of the present invention, there is also provided a method of binding and/or modifying a DNA target site in a cell or cell-free system, the method comprising delivering to the cell or cell-free system any one of the compositions described herein,

In some embodiments, the binding and/or modifying occurs in a eukaryotic cell or prokaryotic cell.

In some embodiments, the mammalian cell is a human cell

In some embodiments, the DNA target site is located within or in proximity to a pathogenic allele of a gene.

In some embodiments, the DNA target site is located in a gene selected from the group consisting of ELANE, CXCR4, EMX, RyR2, KNCQ1, KCNH2, SCN5a, GBA1, GBA2, Rhodopsin, GUCY2D, IMPDH1, FGA, BEST1, PRPH2, KRT5, KRT14, ApoA1, STAT3, STAT1, ADA2, RPS19, SBDS, GATA2, RPE65, LDLR, ANGPTL3, B2M, TRAC, TCF4, TGFBi, PAX6, C3, LRRK2, SARM1, SAMD9, SAMD9L, HAVCR2, CD3E, APLP2, CISH, TIGIT, TNNT2, TNN, MYH7, and HLA-E.

In some embodiments, the DNA target is repaired with an exogenous donor molecule.

In some embodiments, the exogenous donor molecule is an RNA or DNA molecule.

In some embodiments, the off-target editing activity is reduced by at least 2-fold, 10-fold, 10²-fold, 10³-fold, 10⁴-fold, 10⁵-fold, or 10⁶-fold relative to the wild-type nuclease (SEQ ID NO: 1).

According to embodiments of the present invention, there is also provided a modified cell obtained by any one of the methods described herein.

In some embodiments, the cell is capable of engraftment.

In some embodiments, the cell is capable of giving rise to progeny cells after engraftment.

In some embodiments, the cell is capable of giving rise to progeny cells after an autologous engraftment.

In some embodiments, the cell is capable of giving rise to progeny cells for at least 12 months or at least 24 months after engraftment.

In some embodiments, the cell is selected from the group consisting of a hematopoietic stem cell, a progenitor cell, a CD34+ hematopoietic stem cell, a bone marrow cell, and a peripheral mononucleated cell.

According to embodiments of the present invention, there is also provided a composition comprising any one of the modified cells described herein and a pharmaceutically acceptable carrier.

According to embodiments of the present invention, there is also provided an in vitro or ex vivo method of preparing the composition comprising mixing the cells with the pharmaceutically acceptable carrier.

In some embodiments, there is provided a variant of an OMNI-50 nuclease with increased specificity and/or activity and/or stability as compared to the wild-type OMNI-50 nuclease (SEQ ID NO: 1, which is encoded by the polynucleotide set forth as SEQ ID NO: 31), as well as methods of using the improved variants. In some embodiments, the increased specificity is increased fidelity. In some embodiments, the increased specificity is increased discrimination. For example, in some embodiments the OMNI-50 variant nuclease displays increased discrimination when targeting a sequence in a mutant allele over a corresponding functional allele relative to a wild-type OMNI-50 nuclease. In some embodiments, there is provided a variant of an OMNI-50 nuclease with increased activity as compared to the wild-type OMNI-50 nuclease, as well as methods of using the improved variants. In some embodiments, there is provided a variant of an OMNI-50 nuclease with increased stability compared to the wild-type OMNI-50 nuclease. The term “stability” as used herein includes storage stability and stability during use e.g. during a wash process, and reflects the stability of the variant according to the invention as a function of time e.g., how much activity is retained when the variant is kept at various conditions. The stability is influenced by many factors e.g., pH, temperature, specific solutions, detergent composition. Non-limiting examples of increased/improved stability include increased thermal stability/thermostability (i.e., increased ability to withstand high temperatures without degradation), increased half-life under different conditions (different buffers, salt concentrations, pH), a protein which is less prone to aggregation in different buffers and salt concentrations, and higher yield when purifying the protein.

Advantageously, when the engineered variant OMNI-50 nucleases are active in a CRISPR endonuclease system, the CRISPR endonuclease system displays increased stability, activity, and increased specificity relative to a wild-type CRISPR endonuclease system in which a wild-type OMNI-50 nuclease is active. In some embodiments, the increased specificity is increased fidelity and the variant exhibits reduced off-target editing activity relative to a wild-type CRISPR endonuclease system in which a wild-type OMNI-50 nuclease is active. In some embodiments, the increased specificity is increased discrimination and the variant exhibits increased allele specific editing activity relative to a wild-type CRISPR endonuclease system in which a wild-type OMNI-50 nuclease is active. For example, an engineered variant OMNI-50 nuclease may display improved allele-specific discrimination, e.g. specific binding and activity at a target region which contains a heterozygous SNP present in only the targeted allele and not present in the non-targeted allele.

In some embodiments, there is provided a variant of an OMNI-50 nickase with increased stability and/or specificity and/or increased activity as compared to the wild-type OMNI-50 nickase. In some embodiments, there is provided a variant of an OMNI-50 dead nuclease with increased stability and/or specificity and/or increased targeting activity as compared to the wild-type OMNI-50 dead nuclease. For example, the catalytic site of any one of the OMNI-50 nuclease variants provided herein may be modified such that the variant has nickase activity, such that it is capable of performing single-strand DNA cuts. Alternatively, the catalytic site of any one of the OMNI-50 nuclease variants provided herein may be modified such that the variant has no nuclease activity, i.e. a dead nuclease.

According to some embodiments of the present invention, there is provided a variant of OMNI-50 nuclease protein comprising a sequence that is at least 80% identical to the amino acid sequence of wild-type OMNI-50 nuclease protein (SEQ ID NO: 1).

In some embodiments, the invention provides a composition comprising a non-naturally occurring OMNI-50 nuclease variant having at least 80%, 85%, 90%, 95%, or 97% identity to wild-type OMNI-50 protein sequence (SEQ ID NO: 1) comprising an amino acid substitution in at least 1, 2, 3, 4, 5, 6, 7, 8, 9, 10, 11, or 12 of the following positions: N300, G614, N698, E836, T939, L1100, D252, D281, L302, N368, S1339, and S779 compared to SEQ ID NO: 1. Each possibility represents a separate embodiment of the present disclosure, and can be combined at will. Any combination of the amino acids in the above twelve positions may be substituted to a different amino acid an OMNI-50 nuclease variant. Amino acids in positions other than the twelve positions listed above may be substituted as well, such that the OMNI-50 nuclease variant has at least 80%, 85%, 90%, 95%, or 97% identity to wild-type OMNI-50 protein sequence (SEQ ID NO: 1).

In some embodiments, the invention provides a composition comprising a non-naturally occurring OMNI-50 nuclease variant having at least 80%, 85%, 90%, 95%, or 97% identity to wild-type OMNI-50 protein sequence (SEQ ID NO: 1) comprising an amino acid substitution in at least 1, 2, 3, 4, 5, or 6 of the following positions: N300, G614, N698, E836, T939, and L1100 compared to SEQ ID NO: 1. Each possibility represents a separate embodiment of the present disclosure, and can be combined at will.

In some embodiments, the invention provides a composition comprising a non-naturally occurring OMNI-50 nuclease variant having at least 80%, 85%, 90%, 95%, or 97% identity to wild-type OMNI-50 protein sequence (SEQ ID NO: 1) comprising an amino acid substitution in at least 1, 2, 3, 4, 5, or 6 of the following positions: D252, D281, L302, N368, L1100, and S1339, compared to SEQ ID NO: 1. Each possibility represents a separate embodiment of the present disclosure and can be combined at will in any combination.

In some embodiments, the invention provides a composition comprising a non-naturally occurring OMNI-50 nuclease variant having at least 80%, 85%, 90%, 95%, or 97% identity to wild-type OMNI-50 protein sequence (SEQ ID NO: 1) comprising an amino acid substitution in at least 1, 2, 3, 4, 5, 6, 7, or 8 of the following positions: N300, G614, N698, S779, E836, T939, L1100, and S1339, compared to SEQ ID NO: 1. Each possibility represents a separate embodiment of the present disclosure and can be combined at will in any combination.

In some embodiments, the non-naturally occurring OMNI-50 nuclease variant exhibits improved activity fidelity and/or targeting, compared to wild-type OMNI-50 protein (SEQ ID NO: 1). In some embodiments, such variant comprises an amino acid substitution at position L1100 compared to SEQ ID NO: 1. In some embodiments, such variant further comprises a substitution in at least 1, 2, 3, 4, or 5 of the following positions: N300, G614, N698, E836, T939, compared to SEQ ID NO: 1. In some embodiments, such variant further comprises substitution in at least 1, 2, 3, 4, or 5 of the following positions: D252, D281, L302, N368, and S1339, compared to SEQ ID NO: 1. In some embodiments, such variant further comprises a substitution at position S779, compared to SEQ ID NO: 1.

In some embodiments, the non-naturally occurring OMNI-50 nuclease variant exhibits increased specificity, compared to wild-type OMNI-50 protein (SEQ ID NO: 1). In some embodiments, such variant comprises an amino acid substitution at position S1339 compared to SEQ ID NO: 1. In some embodiments, such variant further comprises a substitution in at least 1, 2, 3, 4, 5, or 6 of the following positions: N300, G614, N698, E836, T939, and L1100 compared to SEQ ID NO: 1. In some embodiments, such variant further comprises substitution in at least 1, 2, 3, or 4 of the following positions: D252, D281, L302, and N368, compared to SEQ ID NO: 1. In some embodiments, such variant further comprises a substitution at position S779, compared to SEQ ID NO: 1.

In some embodiments, the non-naturally occurring OMNI-50 nuclease variant exhibits increased thermostability, compared to wild-type OMNI-50 protein (SEQ ID NO: 1). In some embodiments, such variant comprises an amino acid substitution at position S779 compared to SEQ ID NO: 1.

In some embodiments, the invention provides a non-naturally occurring OMNI-50 nuclease variant having a sequence of at least 80%, 85%, 90%, 95%, or 97% identity to wild-type OMNI-50 protein sequence (SEQ ID NO: 1) comprising at least 1, 2, 3, 4, 5, 6, 7, 8, 9, 10, 11, or 12 of the following amino acid substitutions: N300A, G614R, N698L, E836F, T939L, LIl00F, D252Y, D281V, L302N, N368S, S1339R, and S779P, compared to SEQ ID NO: 1. Each possibility represents a separate embodiment of the present disclosure and can be combined at will in any combination.

In some embodiments, the invention provides a non-naturally occurring OMNI-50 nuclease variant having a sequence of at least 80%, 85%, 90%, 95%, or 97% identity to wild-type OMNI-50 protein sequence (SEQ ID NO: 1) comprising at least 1, 2, 3, 4, 5, or 6 of the following amino acid substitutions: N300A, G614R, N698L, E836F, T939L, and L1100F, compared to SEQ ID NO: 1. Each possibility represents a separate embodiment of the present disclosure and can be combined at will in any combination.

In some embodiments, the invention provides a non-naturally occurring OMNI-50 nuclease variant having a sequence of at least 80%, 85%, 90%, 95%, or 97% identity to wild-type OMNI-50 protein sequence (SEQ ID NO: 1) comprising at least 1, 2, 3, 4, 5, or 6 of the following amino acid substitutions: D252Y, D281V, L302N, N368S, L1100F, and S1339R, compared to SEQ ID NO: 1. Each possibility represents a separate embodiment of the present disclosure and can be combined at will in any combination.

In some embodiments, the invention provides a non-naturally occurring OMNI-50 nuclease variant having a sequence of at least 80%, 85%, 90%, 95%, or 97% identity to wild-type OMNI-50 protein sequence (SEQ ID NO: 1) comprising at least 1, 2, 3, 4, 5, 6, 7, or 8 of the following amino acid substitutions: N300A, G614R, N698L, S779P, E836F, T939L, L1100F, and S1339R, compared to SEQ ID NO: 1. Each possibility represents a separate embodiment of the present disclosure and can be combined at will in any combination.

According to some embodiments of the present invention, there is provided a non-naturally occurring OMNI-50 nuclease variant having at least 80%, 85%, 90%, 95%, or 97% identity to a wild-type OMNI-50 protein sequence (SEQ ID NO: 1) comprising an amino acid substitution in at least one of the following positions: N300, G614, N698, S779, E836, T939, L1100, and S1339 compared to SEQ ID NO:1. In some embodiments, there is provided a non-naturally occurring OMNI-50 nuclease variant comprising an amino acid substitution in at least 1, 2, 3, 4, 5, 6, 7 or 8 of the following positions: N300, G614, N698, S779, E836, T939, L1100, and S1339 compared to SEQ ID NO: 1. Each possibility represents a separate embodiment of the disclosure and can be combined at will.

In some embodiments, the non-naturally occurring OMNI-50 nuclease variant comprises an amino acid substitution in position S779 compared to SEQ ID NO:1. In some embodiments, such variant exhibits improved stability compared to a comparable/identical sequence lacking the indicated substitution and/or to wild-type OMNI-50 (SEQ ID NO: 1). In some embodiments, the non-naturally occurring OMNI-50 nuclease variant comprises an amino acid substitution in position S779 and additional 1, 2, 3, 4, 5, 6, or 7 amino acid substitutions in the following positions: N300, G614, N698, E836, T939, L1100, and S1339 compared to SEQ ID NO: 1.

In some embodiments, the non-naturally occurring OMNI-50 nuclease variant comprises an amino acid substitution in position S1339 compared to SEQ ID NO:1. In some embodiments, such variant exhibits improved specificity and/or activity compared to a comparable/identical sequence lacking the indicated substitutions.

In some embodiments, the non-naturally occurring OMNI-50 nuclease variant comprises an amino acid substitution in positions S779 and S1339, and optionally additional 1, 2, 3, 4, 5, or 6 amino acid substitutions in the following positions: N300, G614, N698, E836, T939, and L1100, compared to SEQ ID NO:1. In some embodiments, such variant exhibits improved stability and/or specificity and/or activity compared to a comparable/identical sequence lacking the indicated substitutions.

In some embodiments, the non-naturally occurring OMNI-50 nuclease variant comprises an amino acid substitution in positions S779, L1100, and S1339, and optionally additional 1, 2, 3, 4, or 5, amino acid substitutions in the following positions: N300, G614, N698, E836, and T939, compared to SEQ ID NO:1.

In some embodiments, the amino acid substitution at position S779 is any one of the following substitutions: S779D, S779E, S779R, S779T, S779N, S779Q, S779G, S779P, S779C, S779A, S779V, S779I, S779L, S779M, S779F, S779Y, or S779W. Each possibility represents a separate embodiment of the present disclosure. In some embodiments, the amino acid substitution at position S779 is any one of the following substitution groups: Nonpolar aliphatic (G, A, V, L, M, I); Aromatic (F, Y, W); Positively charged (K, R, H); Polar uncharged (S, T, C, P, N, Q); and Negatively charged (D, E). In some embodiments, the amino acid substitution at position S779 is any one of the following substitutions: alanine (A), aspartic acid (D), asparagine (N), histidine (H), or phenylalanine (F).

In some embodiments, the amino acid substitution at position S1339 is any one of the following substitutions: S1339D, S1339E, S1339R, S1339T, S1339N, S1339Q, S1339G, S1339P, S1339C, S1339A, S1339V, S13391, S1339L, S1339M, S1339F, S1339Y, or S1339W. Each possibility represents a separate embodiment of the present disclosure.

According to some embodiments of the present invention, there is provided a non-naturally occurring OMNI-50 nuclease variant having at least 80%, 85%, 90%, 95%, or 97% identity to wild-type OMNI-50 protein sequence (SEQ ID NO: 1) and comprising an amino acid substitution of at least one of the following: N300A, G614R, N698L, S779P, E836F, T939L, L1100F, and S1339R, compared to SEQ ID NO: 1. In some embodiments, the non-naturally occurring OMNI-50 nuclease variant comprises at least 1, 2, 3, 4, 5, 6, 7, or 8 of the following substitutions: N300A, G614R, N698L, S779P, E836F, T939L, L1100F, and S1339R compared to SEQ ID NO:1. Each possibility represents a separate embodiment of the invention and can be combined at will.

In some embodiments, the variant OMNI-50 nuclease comprises an amino acid sequence of any one of SEQ ID NOs: 2-30.

In some embodiments, there is provided a polynucleotide sequence encoding the non-naturally occurring OMNI-50 nuclease variant, which has a wild-type OMNI-50 protein sequence (SEQ ID NO: 1) comprising an amino acid substitution in at least one of the following positions: N300, G614, N698, E836, T939, L1100, D252, D281, L302, N368, S1339, and S779. In some embodiments, the polynucleotide sequence comprises a nucleotide sequence selected from SEQ ID NOs: 32-60. In some embodiments the polynucleotide is an DNA expression vector. In some embodiments the polynucleotide is an RNA molecule, preferably an mRNA molecule.

According to some embodiments of the present invention, there is provided a CRISPR system comprising any one of the OMNI-50 nuclease variant disclosed herein complexed with a guide RNA molecule that targets a DNA target site, wherein the CRISPR system displays reduced off-target editing activity relative to a wild-type CRISPR system comprising a wild-type OMNI-50 nuclease protein and the guide RNA molecule. In some embodiments, the guide RNA molecule is a single-guide RNA (sgRNA). In some embodiments, the guide RNA molecule is part of a crRNA:tracrRNA complex.

In some embodiments, the OMNI-50 variant nuclease exhibits increased specificity and/or activity to a target site when complexed with a guide RNA targeting the OMNI-50 variant to the target site compared to a wild-type OMNI-50 nuclease (SEQ ID NO: 1).

In some embodiments, the OMNI-50 nuclease variant is a nickase having an inactivated RuvC domain created by an amino acid substitution at a position provided for the CRISPR nuclease in column 1 of the table below. In some embodiments, the nickase further comprises an amino acid substitution in at least one of the following positions: N300, G614, N698, E836, T939, L1100, D252, D281, L302, N368, S1339, and S779.

In some embodiments, the OMNI-50 nuclease variant is a nickase having an inactivated HNH domain created by an amino acid substitution at a position provided for the CRISPR nuclease in column 2 of the table below. In some embodiments, the nickase further comprises an amino acid substitution in at least one of the following positions: N300, G614, N698, E836, T939, L1100, D252, D281, L302, N368, S1339, and S779.

In some embodiments, the OMNI-50 nuclease variant is a catalytically dead nuclease having an inactivated RuvC domain and an inactivated HNH domain created by substitutions at the positions provided for the CRISPR nuclease in column 3 of the table below. In some embodiments, the catalytically dead nuclease comprises an amino acid substitution in at least one of the following positions: N300, G614, N698, E836, T939, L1100, D252, D281, L302, N368, S1339, and S779.

TABLE 1
Positions affecting OMNI-50 nuclease
activity (relative to SEQ ID NO: 1)

| Nickase having | Nickase having | Dead nuclease having |
|---|---|---|
| inactivated RuvC | inactivated HNH | inactivated RuvC and |
| domain | domain | HNH domains |
| D12 or E785 or | D875* or H876 or | (D12 or E785 or H1008 |
| H1008 or D1011 | N899 | or D1011) and (D875* |
| | | or H876 or N899) |

The above table lists alternative positions to be substituted to generate a nickase having an inactivated RUVC domain, alternative positions to be substituted to generate a nickase having an inactivated HNH domain, and alternative positions to be substituted to generate a catalytically dead nuclease having inactivated RUVC and HNH domains. Substitution to any other amino acid is permissible for each of the amino acid positions indicated in columns 1-3, except if followed by an asterisk, which indicates that any substitution other than aspartic acid (D) to glutamic acid (E) or glutamic acid (E) to aspartic acid (D) results in inactivation.

Throughout the text OMNI-50 nuclease variants are referred to, however, any of these variants may be modified to have nickase activity (i.e. nucleases which create a single-strand DNA break as opposed to a double-strand break) or to have no nuclease activity (i.e. a catalytically dead nuclease).

Accordingly, point mutations can be introduced into any one of the variants described herein to modify or abolish their nuclease activity while still retaining their ability to specifically bind DNA in a guide RNA-programmed manner. Any one of these variants can specifically target a desired DNA target sequence via a guide RNA molecule (e.g. a single-guide RNA (sgRNA) or a crRNA:tracrRNA complex). The variant-guide complex will also carry any molecule attached to the complex to the target site. Thus, this disclosure also contemplates fusion proteins comprising any one of the variants described herein and a DNA modifying domain (e.g., a deaminase, a nuclease, a nickase, a recombinase, a methyltransferase, a methylase, an acetylase, an acetyltransferase, a transcriptional activator, or a transcriptional repressor domain), as well as the use of such fusion proteins in correcting mutations in a genome (e.g., the genome of a human subject) that are associated with disease, or generating mutations in a genome (e.g., the human genome) to decrease or prevent expression of a gene.

In some embodiments, any of the variants provided herein may be fused to a protein that has an enzymatic activity. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In some cases, the nuclease activity introduces a double strand break in the target DNA. In some cases, the enzymatic activity modifies a target polypeptide associated with the target DNA. In some cases, the enzymatic activity is methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. In some cases, the target polypeptide is a histone and the enzymatic activity is methyltransferase activity, demethylase activity, acetyltransferase activity, deacetylase activity, kinase activity, phosphatase activity, ubiquitin ligase activity or deubiquitinating activity.

Thus, any one of the OMNI-50 nuclease, nickase, or dead-nuclease variants may be fused (e.g. directly fused or fused via a linker) to another DNA modulating or DNA modifying enzyme, including, but not limited to, base editors such as a deaminase, a reverse transcriptase (e.g. for use in prime editing, see Anzalone et al. (2019)), an enzyme that modifies the methylation state of DNA (e.g. a methyltransferase), or a modifier of histones (e.g. a histone acetyl transferase). Indeed, the OMNI-50 nuclease, nickase, inactive variants described herein may be fused to a DNA modifying enzyme or an effector domain thereof.

Examples of DNA modifiers include but are not limited to: a deaminase, a nuclease, a nickase, a recombinase, a methyltransferase, a methylase, an acetylase, an acetyltransferase, a reverse transcriptase, an helicase, an integrase, a ligase, a transposase, a demethylase, a phosphatase, a transcriptional activator, or a transcriptional repressor. In some embodiments, any of the OMNI-50 variants provided herein are fused to a protein that has an enzymatic activity. In some embodiments, the enzymatic activity modifies a target DNA molecule. The OMNI-50 variants described herein or fusion proteins thereof, may be used to correct or generate one or more mutations in a gene associated with disease, or to increase, correct, decrease, or prevent expression of a gene.

According to some embodiments of the present invention, there is provided a method for gene editing having increased editing activity and/or increased specificity, comprising contacting a DNA target site with an active CRISPR system comprising any one of the OMNI-50 nuclease variant proteins described herein.

According to some embodiments, there is provided a method for gene editing having increased on-target activity and/or increased fidelity (reduced off target activity) and/or increased discrimination (increased allele specific editing), comprising:
contacting a target site locus with an active CRISPR system comprising a variant OMNI-50 nuclease protein of any one of the variants described herein, wherein the active CRISPR system displays reduced off-target editing activity and maintained on-target editing activity relative to a wild-type CRISPR system having a wild-type OMNI-50 nuclease protein.

The present disclosure provides an engineered OMNI-50 nuclease exhibiting increased specificity to a target site compared to the wild-type OMNI-50 nuclease (SEQ ID NO: 1). The wild-type OMNI-50 nuclease is disclosed in PCT International Application Publication No. WO/2020-030782, incorporated herein by reference. When the engineered OMNI-50 nuclease variant is active in a CRISPR endonuclease system, the CRISPR endonuclease system displays reduced off-target editing activity and/or increased editing activity and/or increased discrimination maintained on-target editing activity relative to a CRISPR endonuclease system comprising the wild-type OMNI-50 nuclease. In some embodiments, the engineered OMNI-50 nuclease is an OMNI-50 nuclease variant comprising at least one amino acid substitution relative to the wild-type OMNI-50 nuclease. In some embodiments, the engineered OMNI-50 nuclease comprises multiple amino acid substitutions compared to wild-type OMNI-50 nuclease.

In some embodiments, OMNI-50 nuclease variant is at least 80%, e.g., at least 85%, 86% 87%, 88%, 89%, 90%, 91%, 92% 93%, 94%, 95%, 96%, 97%, 98% or 99% identical to the amino acid sequence of SEQ ID NO: 1. As a non-limiting example, an OMNI-50 nuclease variant may have amino acid sequence differences at up to 1%, 2%, 3%, 4%, 5%, 6%, 7%, 8%, 9%, 10%, 11%, 12%, 13%, 14%, 15%, or 20% of its residues relative to SEQ ID NO: 1. Such sequence differences may be revealed by a sequence alignment. An OMNI-50 variant nuclease may be generated by replacing at least one amino acid residue of an OMNI-50 wild-type nuclease with another amino acid residue e.g. with a conservative or non-conservative amino acid substitution, and/or by inserting or deleting an amino acid residue of the OMNI-50 wild-type nuclease. Any such mutations, including but not limited to substitutions, insertions, or deletions, in addition to any other mutations described herein, or with mutations in addition to the mutations described herein, may be used to generate an OMNI-50 variant nuclease from an OMNI-50 wild-type nuclease. In some embodiments, the OMNI-50 variant nuclease retains a desired activity of the parent wild-type OMNI-50 nuclease, e.g., the ability to interact with a guide RNA and target DNA and/or the activity of the nuclease (e.g., ability to cause a double-strand DNA break, a single-strand DNA break, or lack of any nuclease or nickase activity). In some embodiments, the variant exhibits increased activity to that of the parent, e.g., nuclease activity, at a level greater than the level of activity of the parent. In some embodiments, the variant retains the desired activity of the parent, e.g. nuclease activity, at a level greater than or equal to the level of activity of the parent. In some embodiments, the variant retains the desired activity of the parent at a level of at least 100%, 95%, 90%, 80%, 70%, 60%, 50%, 40%, or 30% the level of activity of the parent. In some embodiments, the OMNI-50 variant nuclease displays reduced off-target effects relative to OMNI-50 wild-type nuclease.

In some embodiments, there is provided a variant of OMNI-50 nuclease protein comprising a sequence that is at least 80% identical to the amino acid sequence of wild-type OMNI-50 (SEQ ID NO: 1) and having at least one amino acid substitution. In some embodiments, the amino acid substitution comprises an amino acid residue replacement to a positive, negative, uncharged, hydrophilic, hydrophobic, polar, or non-polar amino acid. In some embodiments, the amino acid substitution is selected from replacement of an amino acid to any one of a different amino acid selected from the group consisting of R, K, H, D, E, S, T, N, Q, C, U, G, P, A, I, L, M, F, W, Y and V.

Positive amino acids include any amino acid having a positively charged R-group, e.g. lysine (K), arginine (R), or Histidine (H). Negative amino acids include any amino acid having a negatively charged R-group, e.g. aspartic acid (D) or glutamic acid (E).

Uncharged amino acids or neutral amino acids include amino acids whose R-group does not normally carry a charge. Polar amino acids include any amino acid having a polar R-group, e.g. serine (S), threonine (T), tyrosine (Y), asparagine (N), or glutamine (Q). Non-polar amino acids include any amino acid having a non-polar R-group, e.g. glycine (G), alanine (A), valine (V), cysteine (C), proline (P), leucine (L), isoleucine (I), methionine (M), tryptophan (W), or phenylalanine (F).

Large hydrophobic amino acid include leucine, methionine, proline, and valine. Aromatic amino acids include histidine, phenylalanine, tryptophan and tyrosine. Polar uncharged amino acid include serine, cysteine, threonine, asparagine, and glutamine.

Properties of an original variant protein having an original amino acid substitution at a given position may be extended to a different variant having a different amino acid substitution at the same position if the different amino acid substitution has an R-group with similar properties to the original amino acid substitution. For example, if a variant protein is shown to have higher specificity compared to a wild-type protein by substituting a glutamic acid (E) residue for a lysine (K) residue, it is reasonable to consider that a similar variant substituting the glutamic acid (E) residue for an arginine (R) residue will also display higher specificity since both lysine (K) and arginine (R) share similar properties (e.g. they both contain positively charged R-groups). Conversely, a variant having a substitution of the glutamic acid (E) residue to an aspartic acid (D) is less likely to display the higher specificity property because both glutamic acid (E) and aspartic acid (D) share the similar property of both containing a negatively charged R-group.

In some embodiments, there is provided a variant of OMNI-50 nuclease protein comprising a sequence that is at least 80% identical to the amino acid sequence of the wild-type OMNI-50 nuclease (SEQ ID NO: 1) and having at least one amino acid substitution in at least one of the following positions in the wild-type OMNI-50 protein sequence: N300, G614, N698, E836, T939, L1100, D252, D281, L302, N368, S1339, and S779. In some embodiments, a variant OMNI-50 nuclease protein contains an amino acid substitution in at least one of the following positions in the wild-type OMNI-50 protein sequence (SEQ ID NO: 1): N300, G614, N698, E836, T939, L1100, D252, D281, L302, N368, S1339, and S779. Each possibility represents a separate embodiment of the present disclosure.

In some embodiments, the variant OMNI-50 nuclease protein comprises at least one amino acid substitution in the following positions in the double-strand DNA nuclease catalytically active form, nickase, or catalytically dead form of OMNI-50 protein sequence: N300, G614, N698, E836, T939, L1100, D252, D281, L302, N368, S1339, and S779. In some embodiments, the variant OMNI-50 nuclease protein comprises at least one of the following amino acid substitutions in the following positions in the fully active, nickase, or catalytically dead form of the wild-type OMNI-50 protein sequence: N300A, G614R, N698L, E836F, T939L, L1100F, D252Y, D281V, L302N, N368S, S1339R, and S779P.

In some embodiments, the variant OMNI-50 nuclease protein comprises at least one amino acid substitution in the following positions in the double-strand DNA nuclease catalytically active form, nickase, or catalytically dead form of OMNI-50 protein sequence: N300, G614, N698, S779, E836, T939, L1100, and S1339. In some embodiments, the variant OMNI-50 nuclease protein comprises at least one of the following amino acid substitutions in the following positions in the fully active, nickase, or catalytically dead form of the wild-type OMNI-50 protein sequence: N300A, G614R, N698L, S779P, E836F, T939L, L1100F, and S1339R.

In some embodiments, the OMNI-50 variant nuclease further comprises one or more of a nuclear localization sequence (NLS), cell penetrating peptide sequence, and/or affinity tag (e.g., HIS-tag, HA-tag). In an embodiment, the OMNI-50 variant nuclease comprises one or more nuclear localization sequences of sufficient strength to drive accumulation of a CRISPR complex comprising the CRISPR nuclease in a detectable amount in the nucleus of a eukaryotic cell. Examples of OMNI-50 variant nuclease sequences engineered to comprise additional peptides are provided by SEQ ID NOs: 61-89. Specifically, these examples provide variants engineered in the following format: NLS-Variant-HA-NLS-8xHis. Polynucleotides encoding the amino acids of SEQ ID NOs: 61-89 are provided by SEQ ID NOs: 90-118, respectively.

According to some embodiments, there is provided an isolated OMNI-50 variant nuclease protein comprising one or more substitutions or mutations relative to the wild-type OMNI-50 nuclease sequence, wherein the isolated variant OMNI-50 variant nuclease is active in a CRISPR system, wherein the CRISPR system displays reduced off-target editing activity and maintained on-target editing activity relative to a wild-type CRISPR system.

According to some embodiments, additional mutations to the OMNI-50 variant nuclease described herein may be implemented. Examples include, but are not limited to, mutations which alter the PAM recognition sequence, alter the nuclease activity of the enzyme, and truncations or removal of portions of the nuclease. According to some embodiments, the variant OMNI-50 variant nuclease may be encoded by any nucleic acid sequence which produces the desired amino acid sequence of the variant. For example, the nuclei acid sequence may be codon-optimized for a cell, such as a bacterial cell, plant cell, or mammalian cell.

In embodiments of the present invention, a CRISPR nuclease and a targeting molecule form a CRISPR complex that binds to a target DNA sequence to effect cleavage of the target DNA sequence. A CRISPR nuclease may form a CRISPR complex comprising the CRISPR nuclease and a single-guide RNA (sgRNA) molecule. Alternatively, a CRISPR nucleases may form a CRISPR complex comprising the CRISPR nuclease, an crRNA molecule, and a tracrRNA molecule. In some embodiments the single-guide RNA (sgRNA) molecule comprises an RNA sequence having at least 80%, 85%, 90%, 95% identity to a sequence as set forth in SEQ ID NO: 140 or SEQ ID NO: 141.

According to some embodiments of the present invention, there is provided a method of gene editing having reduced off-target editing activity and/or increased on-target editing activity, comprising: contacting a target site locus with an active CRISPR endonuclease system having a variant OMNI-50 protein complexed with a suitable guide RNA or guide RNA complex, wherein the active CRISPR endonuclease system displays increased editing activity and/or reduced off-target editing activity and or increased allele specific editing relative to a wild-type OMNI-50 CRISPR system.

According to some embodiments of the present invention, there is provided a method for gene editing having reduced off-target editing activity, comprising contacting a DNA target site with an active CRISPR system comprising any one of the OMNI-50 nuclease variant proteins described herein. According to some embodiments of the present invention, there is provided a method for gene editing having increased editing activity, comprising contacting a DNA target site with an active CRISPR system comprising any one of the OMNI-50 nuclease variant proteins described herein. According to some embodiments of the present invention, there is provided a method for gene editing having increased allele specific editing, comprising contacting a DNA target site with an active CRISPR system comprising any one of the OMNI-50 nuclease variant proteins described herein.

In some embodiments, the gene editing occurs in a eukaryotic cell or prokaryotic cell. In some embodiments, the eukaryotic cell is a plant cell or mammalian cell. In some embodiments, the mammalian cell is a human cell. In some embodiments, the DNA target site is located within or in proximity to a pathogenic allele of a gene.

In some embodiments, the DNA target site is located in a gene selected from the group consisting of: ELANE, CXCR4, EMX, RyR2, KNCQ1, KCNH2, SCN5a, GBA1, GBA2, Rhodopsin, GUCY2D, IMPDH1, FGA, BEST1, PRPH2, KRT5, KRT14, ApoA1, STAT3, STAT1, ADA2, RPS19, SBDS, GATA2, RPE65, LDLR, ANGPTL3, B2M, TRAC, TCF4, TGFBi, PAX6, C3, LRRK2, SARM1, SAMD9, SAMD9L, HAVCR2, CD3E, APLP2, CISH, TIGIT, TNNT2, TNN, MYH7, and HLA-E.

A non-limiting example of a sgRNA sequence that can be utilized to target TRAC includes:

(SEQ ID NO: 142)
UUAGAGUCUCUCAGCUGGUACAGUUUGAGAGUUAUGAAAAUGACGAGU
UCAAAUAAAAAUUUAUUCAAACCGCCUAUUUAUAGGCCGCAGAUGUUC
UGCUUU.

In some embodiments, the DNA target is repaired with an exogenous donor molecule.

In some embodiments, the DNA target site is located in or adjacent to a gene selected from the group consisting of: AAVS1, ABCD1, APOLDI, AQP4, ATP7B, B2M, BCL11A, CCL4, CCR5, CD34, CD52, CD5, CD7, CIITA, CLK3, CLYBL, CTNS, CUL3, DYRK1A, EGFR, EMX1, F8, FANCF, FKTN, GALNS, GRN2B, HAS3, HBB, HPRT1, KRAS, MECP2, MIP, NRL, OMP, OTC, PAH, PDCD1, PDGFRA, PLP1, PPP2R5D, PTEN, RELN, RUNX1, RYR2, SHANK3, SNCA, TMEM175, TRAC, TRBC1, UBE3A, VEGFA, ZSCAN, FBL, FUS, G3BP1, HIST1H2BJ, LAMP1, MAP1LC3B, NPM1, Rab11A, RAD21, Roji1, Roji2, SEC61B, SMC1A, Sqstm1, TOMM20, TOP2a, TUBalB.

In some embodiments, the allele specific editing activity of the variant is increased by at least 1.25-fold, 1.5-fold, 2-fold, 10-fold, 10²-fold, 10³-fold, 10⁴-fold, 10⁵-fold, or 10⁶-fold relative to a wild-type OMNI-50 nuclease. In some embodiments, the off-target editing activity is reduced by at least 2-fold, 10-fold, 10²-fold, 10³-fold, 10⁴-fold, 10⁵-fold, or 10⁶-fold. In some embodiments, the on-target editing activity is increased by at least 1.25-fold, 1.5-fold, 2-fold, 10-fold, 10²-fold, 10³-fold, 10⁴-fold, 10⁵-fold, or 10⁶-fold.

According to some embodiments of the present invention, there is provided a polynucleotide molecule encoding any one of the OMNI-50 variant proteins described herein.

Additional description of the OMNI-50 nuclease (SEQ ID NO: 1) is provided in PCT International Application Publication Nos. WO 2020/223514 A2, WO 2022/098693 A1, and WO 2023/019263A1, the contents of each of which are hereby incorporated by reference.

### Delivery

The OMNI-50 variant compositions described herein may be delivered as a protein, DNA molecules, RNA molecules, Ribonucleoproteins (RNP), nucleic acid vectors, or any combination thereof. In some embodiments, the RNA molecule comprises a chemical modification, Non-limiting examples of suitable chemical modifications include 2′-O-methyl (M), 2′-O-methyl, 3′phosphorothioate (MS) or 2′-O-methyl, 3′thioPACE (MVISP), pseudouridine, and 1-methyl pseudo-uridine. Each possibility represents a separate embodiment of the present invention.

The OMNI-50 variants and/or polynucleotides encoding same described herein, and/or additional molecules, such as a single-guide RNA molecule, crRNA molecule, tracrRNA molecules or a nucleotide molecule that encodes any one of them, may be delivered to a target cell by any suitable means. The target cell may be any type of cell e.g., eukaryotic or prokaryotic, in any environment e.g., isolated or not, maintained in culture, in vitro, ex vivo, in vivo or in planta. A target site in a target cell may be within the nucleus of the cell.

The compositions described herein may be introduced into a cell as part of a vector molecule having additional sequences such as, for example, replication origins, promoters and genes encoding antibiotic resistance. Moreover, compositions may introduced into a cell as naked nucleic acids or proteins, as nucleic acids or proteins complexed with or packaged within an agent such as a liposome, exosome, or poloxamer, or can be delivered by recombinant viruses (e.g., adenovirus, AAV, herpesvirus, retrovirus, lentivirus and integrase defective lentivirus (IDLV)) or virus-like particles. As non-limiting examples, the composition may be packaged into an adeno-associated virus (AAV), or into a lentivirus, such as a non-integrating lentivirus or a lentivirus lacking reverse transcription capability. Additional non-limiting examples include packaging the composition into liposomes, extracellular vesicles, or exosomes, which may be pseudotyped with vesicular stomatitis glycoprotein (VSVG) or conjugated to a cell-penetrating peptide, an antibody, a targeting moiety, or any combination thereof.

In some embodiments, the composition to be delivered includes mRNA of the nuclease and RNA of the guide. In some embodiments, the composition to be delivered includes mRNA of the nuclease, RNA of the guide and a donor template. In some embodiments, the composition to be delivered includes the CRISPR nuclease and guide RNA. In some embodiments, the composition to be delivered includes the CRISPR nuclease, guide RNA and a donor template for gene editing via, for example, homology directed repair. Optionally the lentivirus includes mRNA of the nuclease and a guide RNA molecule, e.g. a single-guide RNA molecule or crRNA molecule, which is used to target the nuclease to a target site. In some embodiments, the composition delivered to a cell includes mRNA of the nuclease, a guide RNA molecule and a donor template molecule. Optionally, the lentivirus includes the nuclease protein variant and a guide RNA molecule. Optionally, the composition delivered to a cell includes the nuclease protein variant, a guide RNA molecule and/or donor template for homology directed repair. Optionally, the composition delivered to a cell includes mRNA of the nuclease variant, a DNA-targeting crRNA molecule, and a tracrRNA molecule. the composition delivered to a cell includes mRNA of the nuclease variant, DNA-targeting crRNA molecule, and a tracrRNA molecule, and a donor template molecule. the composition delivered to a cell includes the nuclease protein variant, DNA-targeting crRNA molecule, and a tracrRNA molecule. Optionally, the composition delivered to a cell includes the nuclease protein variant, DNA-targeting crRNA molecule, and a tracrRNA molecule, and DNA donor template molecule for homology directed repair.

Any suitable viral vector system may be used to deliver such compositions. Conventional viral and non-viral based gene transfer methods can be used to introduce nucleic acids and/or OMNI-50 variant protein in cells (e.g., mammalian cells, plant cells, etc.) and target tissues. Such methods can also be used to administer nucleic acids encoding and/or OMNI-50 variant protein to cells in vitro. In certain embodiments, nucleic acids and/or a OMNI-50 variant protein are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds.) (1995); and Yu et al., Gene Therapy 1:13-26 (1994).

Methods of non-viral delivery of nucleic acids and/or proteins include electroporation, lipofection, microinjection, biolistics, particle gun acceleration, virosomes, virus-like particles, exosomes, liposomes, immunoliposomes, polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. Trends Plant Sci. (2006). Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al., Nat. Biotechnol. (2015), Coelho et al., N. Engl. J. Med. (2013); Judge et al., Mol. Ther. (2006); and Basha et al., Mol. Ther. (2011).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofectionis described in e.g., U.S. Pat. Nos. 5,049,386, 4,946,787; and 4,897,355) and lipofection reagents are sold commercially (e.g., Transfectam.TM., Lipofectin.TM. and Lipofectamine.TM. RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al (2009) Nature Biotechnology 27(7) p. 643).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues. An OMNI-50 variant or a nucleic acid expressing the variant, as well as any associated nucleic acids, may be delivered by a non-integrating lentivirus. Optionally, RNA delivery with lentivirus is utilized. Optionally, the lentivirus includes mRNA of the nuclease and a guide RNA molecule, e.g. a single-guide RNA molecule or crRNA molecule, which is used to target the nuclease to a target site. Optionally the lentivirus includes mRNA of the nuclease, guide RNA molecule and a donor template molecule. Optionally, the lentivirus includes the nuclease protein variant and a guide RNA molecule. Optionally, the lentivirus includes the nuclease protein variant, a guide RNA molecule and/or donor template molecule for homology directed repair. Optionally, the lentivirus includes mRNA of the nuclease variant, a DNA-targeting crRNA molecule, and a tracrRNA molecule. Optionally the lentivirus includes mRNA of the nuclease variant, DNA-targeting crRNA molecule, and a tracrRNA molecule, and a donor template molecule. Optionally, the lentivirus includes the nuclease protein variant, DNA-targeting crRNA molecule, and a tracrRNA molecule. Optionally, the lentivirus includes the nuclease protein variant, DNA-targeting crRNA molecule, and a tracrRNA molecule, and DNA donor template molecule for homology directed repair.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO2013/014537, WO2014/016690, WO2016185125, WO2017194902, and WO2017194903.

The tropism of a retrovirus can be altered by incorporating foreign envelope proteins, expanding the potential target population of target cells. Lentiviral vectors are retroviral vectors capable of transducing or infecting non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher Panganiban, J. Virol. (1992); Johann et al., J. Virol. (1992); Sommerfelt et al., Virol. (1990); Wilson et al., J. Virol. (1989); Miller et al., J. Virol. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood (1995); Kohn et al., Nat. Med. (1995); Malech et al., PNAS (1997)). PA317/pLASN was the first therapeutic vector used in a gene therapy trial. (Blaese et al., Science (1995)). Transduction efficiencies of 50% or greater have been observed for MFG-S packaged vectors. (Ellem et al., Immunol Immunother. (1997); Dranoff et al., Hum. Gene Ther. (1997).

Packaging cells are used to form virus particles that are capable of infecting a host cell. Such cells include 293 cells, which package adenovirus, AAV, and .psi.2 cells or PA317 cells, which package retrovirus. Viral vectors used in gene therapy are usually generated by a producer cell line that packages a nucleic acid vector into a viral particle.

The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art.

In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney et al., Culture of Animal Cells, A Manual of Basic Technique (3^{rd} ed. 1994)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOKISV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with nuclease systems (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-.gamma. and TNF-alpha are known (as a non-limiting example see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panB cells), GR-1 (granulocytes), and lad (differentiated antigen presenting cells) (as a non-limiting example see Inaba et al., J. Exp. Med. 176:1693-1702 (1992)). Stem cells that have been modified may also be used in some embodiments.

Notably, any one of the OMNI-50 variant described herein may be suitable for genome editing in post-mitotic cells or any cell which is not actively dividing, e.g., arrested cells. Examples of post-mitotic cells which may be edited using an OMNI-50 variant of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17^{th} ed., 1989).

### DNA Repair by Homologous Recombination

In some embodiments of the present invention, a variant OMNI-50 nuclease is utilized to affect a DNA break at a target site to induce cellular repair mechanisms, for example, but not limited to, non-homologous end-joining (NHEJ) or homology-directed repair (HDR).

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position. In some embodiment, the nucleic acid template comprises a ribonucleotide sequence, e.g., of one or more ribonucleotides, that corresponds to wild type sequence of the target nucleic acid, e.g., of the target position. In some embodiment, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang and Wilson, Proc. Natl. Acad. Sci. USA (1987); Nehls et al., Science (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention using a donor template for repair may use a DNA or RNA, single-stranded and/or double-stranded donor template that can be introduced into a cell in linear or circular form. In embodiments of the present invention a gene-editing composition comprises: (1) an RNA molecule comprising a guide sequence to affect a double strand break in a gene prior to repair and (2) a donor RNA template for repair, and the RNA molecule comprising the guide sequence is a first RNA molecule and the donor RNA template is a second RNA molecule. In some embodiments, the guide RNA molecule and template RNA molecule are connected as part of a single molecule.

A donor sequence may also be an oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to “correct” a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with or packaged within an agent such as a liposome, exosome, or poloxamer, or can be delivered by recombinant viruses (e.g., adenovirus, AAV, herpesvirus, retrovirus, lentivirus and integrase defective lentivirus (IDLV)) or virus-like particles. Non-viral vectors, such as transposon-based systems, e.g. recombinant Sleeping Beauty transposon systems or recombinant PiggyBac transposon systems, may also be utilized for transposition of a polynucleotide sequence in a target cell.

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPP1R12c (also known as AAVS1) gene, an albumin gene or a Rosa gene. See, e.g., U.S. Pat. Nos. 7,951,925 and 8,110,379; U.S. Publication Nos. 2008/0159996; 20100/0218264; 2010/0291048; 2012/0017290; 2011/0265198; 2013/0137104; 2013/0122591; 2013/0177983 and 2013/0177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA.

### DNA-Targeting RNA Molecules

In embodiments of the present invention, the DNA-targeting RNA sequence comprises a guide sequence portion. The “guide sequence portion” of an RNA molecule refers to a nucleotide sequence that is capable of hybridizing to a specific target DNA sequence, e.g., the guide sequence portion has a nucleotide sequence which is fully complementary to the DNA sequence being targeted along the length of the guide sequence portion. In some embodiments, the guide sequence portion is 17, 18, 19, 20, 21, 22, 23, 24, 25, 26, 27, 28, 29 or 30 nucleotides in length, or approximately 17-30, 17-29, 17-28, 17-27, 17-26, 27-25, 17-24, 18-22, 19-22, 18-20, 17-20, or 21-22 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, the RNA molecule is capable of targeting the CRISPR nuclease to the specific target DNA sequence. Each possibility represents a separate embodiment. An RNA molecule can be custom designed to target any desired sequence.

According to some aspects of the invention, the disclosed methods comprise a method of modifying a nucleotide sequence at a target site in a cell-free system or the genome of a cell comprising introducing into the cell the composition of any one of the embodiments described herein.

In some embodiments, the cell is a eukaryotic cell, preferably a mammalian cell or a plant cell. In some embodiments, genome modifying occurs within the nucleus of a cell.

According to some aspects of the invention, the disclosed methods comprise a use of any one of the compositions described herein for the treatment of a subject afflicted with a disease associated with a genomic mutation comprising modifying a nucleotide sequence at a target site in the genome of the subject.

According to some aspects of the invention, the disclosed methods comprise a method of treating subject having a mutation disorder comprising targeting any one of the compositions described herein to an allele associated with the mutation disorder.

In some embodiments, the mutation disorder is related to a disease or disorder selected from any of a neoplasia, age-related macular degeneration, schizophrenia, neurological, neurodegenerative, or movement disorder, Fragile X Syndrome, secretase-related disorders, prion-related disorders, ALS, addiction, autism, Alzheimer's Disease, neutropenia, inflammation-related disorders, Parkinson's Disease, blood and coagulation diseases and disorders, beta thalassemia, sickle cell anemia, cell dysregulation and oncology diseases and disorders, inflammation and immune-related diseases and disorders, metabolic, liver, hypercholesteremia, kidney and protein diseases and disorders, muscular and skeletal diseases and disorders, dermatological diseases and disorders, neurological and neuronal diseases and disorders, pulmonary disease and disorders, corneal disease and disorders, retinal diseases and disorders, and ocular diseases and disorders.

### Diseases and Therapies

Certain embodiments of the invention target a nuclease to a specific genetic locus associated with a disease or disorder as a form of gene editing, method of treatment, or therapy. For example, to induce editing or knockout of a gene, a novel nuclease disclosed herein may be specifically targeted to a pathogenic mutant allele of the gene using a custom designed guide RNA molecule. The guide RNA molecule is preferably designed by first considering the PAM requirement of the nuclease, which as shown herein is also dependent on the system in which the gene editing is being performed. For example, a guide RNA molecule designed to target an OMNI-50 nuclease to a target site is designed to contain a spacer sequence complementary to a DNA strand of a DNA double-stranded region that neighbors a OMNI-50 PAM sequence, e.g. “NGG.” The guide RNA molecule is further preferably designed to contain a spacer region (i.e. the region of the guide RNA molecule having complementarity to the target allele) of sufficient and preferably optimal length in order to increase specific activity of the nuclease and reduce off-target effects.

As a non-limiting example, the guide RNA molecule may be designed to target the nuclease to a specific region of a mutant allele, e.g. near the start codon, such that upon DNA damage caused by the nuclease a non-homologous end joining (NHEJ) pathway is induced and leads to silencing of the mutant allele by introduction of frameshift mutations. This approach to guide RNA molecule design is particularly useful for altering the effects of dominant negative mutations and thereby treating a subject. As a separate non-limiting example, the guide RNA molecule may be designed to target a specific pathogenic mutation of a mutated allele, such that upon DNA damage caused by the nuclease a homology directed repair (HDR) pathway is induced and leads to template mediated correction of the mutant allele. This approach to guide RNA molecule design is particularly useful for altering haploinsufficiency effects of a mutated allele and thereby treating a subject.

Non-limiting examples of specific genes which may be targeted for alteration to treat a disease or disorder are presented herein below. Specific disease-associated genes and mutations that induce a mutation disorder are described in the literature. Such mutations can be used to design a DNA-targeting RNA molecule to target a CRISPR composition to an allele of the disease associated gene, where the CRISPR composition causes DNA damage and induces a DNA repair pathway to alter the allele and thereby treat the mutation disorder.

Mutations in the ELANE gene are associated with neutropenia. Accordingly, without limitation, embodiments of the invention that target ELANE may be used in methods of treating subjects afflicted with neutropenia. Guide RNA molecules which target the ELANE gene and are useful for treating neutropenia are disclosed in PCT International Application No. PCT/US2020/059186, incorporated herein by reference.

CXCR4 is a co-receptor for the human immunodeficiency virus type 1 (HIV-1) infection. Accordingly, without limitation, embodiments of the invention that target CXCR4 may be used in methods of treating subjects afflicted with HIV-1 or conferring resistance to HIV-1 infection in a subject.

Programmed cell death protein 1 (PD-1) disruption enhances CAR-T cell mediated killing of tumor cells and PD-1 may be a target in other cancer therapies. Accordingly, without limitation, embodiments of the invention that target PD-1 may be used in methods of treating subjects afflicted with cancer. In an embodiment, the treatment is CAR-T cell therapy with T cells that have been modified according to the invention to be PD-1 deficient.

In addition, BCL11A is a gene that plays a role in the suppression of hemoglobin production. Globin production may be increased to treat diseases such as thalassemia or sickle cell anemia by inhibiting BCL11A. See for example, PCT International Publication No. WO 2017/077394A2; U.S. Publication No. US2011/0182867A1; Humbert et al. Sci. Transl. Med. (2019); and Canver et al. Nature (2015). Accordingly, without limitation, embodiments of the invention that target an enhancer of BCL11A may be used in methods of treating subjects afflicted with beta thalassemia or sickle cell anemia.

Embodiments of the invention may also be used for targeting any disease-associated gene, for studying, altering, or treating any of the diseases or disorders listed in Table A or Table B below. Indeed, any disease-associated with a genetic locus may be studied, altered, or treated by using the nucleases disclosed herein to target the appropriate disease-associated gene, for example, those listed in U.S. Publication No. 2018/0282762A1 and European Patent No. EP3079726B1.

TABLE A
Diseases, Disorders and their associated genes

| DISEASE/ | |
|---|---|
| DISORDERS | GENE(S) |
| Neoplasia | PTEN; ATM; ATR; EGFR; ERBB2; ERBB3; ERBB4; |
| | Notch1; Notch2; Notch3; Notch4; AKT; AKT2; AKT3; |
| | HIF; HIF1a; HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); gf2 (3 variants); Igf 1 Receptor; Igf 2 Receptor; |
| | Bax; Bcl2; caspases family (9 members: 1, 2, 3, 4, 6, 7, 8, |
| | 9, 12); Kras; Apc |
| Age-related Macular | Abcr; Ccl2; Cc2; cp (ceruloplasmin); Timp3; cathepsinD; |
| Degeneration | Vldlr; Ccr2 |
| Schizophrenia | Neuregulin1 (Nrg1); Erb4 (receptor for Neuregulin); |
| | Complexin1 (Cp1x1); Tph1 Tryptophan hydroxylase; Tph2 |
| | Tryptophan hydroxylase 2; Neurexin 1; GSK3; GSK3a; |
| | GSK3b |
| Neurological, Neuro | 5-HTT (S1c6a4); COMT; DRD (Drd1a); SLC6A3; DAOA; |
| degenerative, and | DTNBP1; Dao (Dao1) |
| Movement Disorders | |
| Trinucleotide Repeat | HTT (Huntington's Dx); SBMA/SMAX1/AR (Kennedy's |
| Disorders | Dx); FXN/X25 (Friedrich's Ataxia); ATX3 (Machado- |
| | Joseph's Dx); ATXN1 and ATXN2 (spinocerebellar |
| | ataxias); DMPK (myotonic dystrophy); Atrophin-1 and |
| | Atn1 (DRPLA Dx); CBP (Creb-BP - global instability); |
| | VLDLR (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin |
| Disorders | (Ncstn); PEN-2 |
| Others | Nos1; Parp1; Nat1; Nat2 |
| Prion related disorders | Prp |
| ALS | SOD1; ALS2; STEX; FUS; TARDBP; VEGF (VEGF-a; |
| | VEGF-b; VEGF-c) |
| Addiction | Prkce (alcohol); Drd2; Drd4; ABAT (alcohol); GRIA2; |
| | Grm5; Grin1; Htr1b; Grin2a; Drd3; Pdyn; Gria1 (alcohol) |
| Autism | Mecp2; BZRAP1; MDGA2; Sema5A; Neurexin 1; Fragile |
| | X (FMR2 (AFF2), FXR1; FXR2; Mglur5) |
| Alzheimer's Disease | E1; CHIP; UCH; UBB; Tau; LRP; PICALM; Clusterin; |
| | PS1; SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); |
| | IL-17b; IL-17c; IL-17d; IL-17f); II-23; Cx3cr1; ptpn22; |
| | TNFa; NOD2/CARD15 for IBD; IL-6; IL-12 (IL-12a; IL- |
| | 12b); CTLA4; Cx3cl1 |
| Parkinson's Disease | x-Synuclein; DJ-1; LRRK2; Parkin; PINK1 |

TABLE B
Diseases, Disorders and their associated genes

| DISEASE CATEGORY | DISEASE AND ASSOCIATED GENES |
|---|---|
| Blood and coagulation | Anemia (CDAN1, CDA1, RPS19, DBA, PKLR, PK1, |
| diseases and disorders | NT5C3, UMPH1, PSN1, RHAG, RH50A, NRAMP2, |
| | SPTB, ALAS2, ANH1, ASB, ABCB7, ABC7, ASAT); |
| | Bare lymphocyte syndrome (TAPBP, TPSN, TAP2, |
| | ABCB3, PSF2, RING11, MHC2TA, C2TA, RFX5, |
| | RFXAP, RFX5), Bleeding disorders (TBXA2R, P2RX1, |
| | P2X1); Factor H and factor H-like 1 (HF1, CFH, HUS); |
| | Factor V and factor VIII (MCFD2); Factor VII deficiency |
| | (F7); Factor X deficiency (F10); Factor XI deficiency |
| | (F11); Factor XII deficiency (F12, HAF); Factor XIIIA |
| | deficiency (F13A1, F13A); Factor XIIIB deficiency |
| | (F13B); Fanconi anemia (FANCA, FACA, FA1, FA, FAA, |
| | FAAP95, FAAP90, FLJ34064, FANCB, FANCC, FACC, |
| | BRCA2, FANCD1, FANCD2, FANCD, FACD, FAD, |
| | FANCE, FACE, FANCF, XRCC9, FANCG, BRIP1, |
| | BACH1, FANCJ, PHF9, FANCL, FANCM, KIAA1596); |
| | Hemophagocytic lymphohistiocytosis disorders (PRF1, |
| | HPLH2, UNC13D, MUNC13-4, HPLH3, HLH3, FHL3); |
| | Hemophilia A (F8, F8C, HEMA); Hemophilia B (F9, |
| | HEMB), Hemorrhagic disorders (PI, ATT, F5); Leukocyde |
| | deficiencies and disorders (ITGB2, CD18, LCAMB, LAD, |
| | EIF2B1, EIF2BA, EIF2B2, EIF2B3, EIF2B5, LVWM, |
| | CACH, CLE, EIF2B4); Sickle cell anemia (HBB); |
| | Thalassemia (HBA2, HBB, HBD, LCRB, HBA1) |
| Cell dysregulation and | B-cell non-Hodgkin lymphoma (BCL7A, BCL7); |
| oncology diseases and | Leukemia (TAL1, TCL5, SCL, TAL2, FLT3, NBS1, NBS, |
| disorders | ZNFN1A1, IK1, LYF1, HOXD4, HOX4B, BCR, CML, |
| | PHL, ALL, ARNT, KRAS2, RASK2, GMPS, AF10, |
| | ARHGEF12, LARG, KIAA0382, CALM, CLTH, CEBPA, |
| | CEBP, CHIC2, BTL, FLT3, KIT, PBT, LPP, NPM1, |
| | NUP214, D9S46E, CAN, CAIN, RUNX1, CBFA2, AML1, |
| | WHSC1L1, NSD3, FLT3, AF1Q, NPM1, NUMA1, |
| | ZNF145, PLZF, PML, MYL, STAT5B, AF10, CALM, |
| | CLTH, ARL11, ARLTS1, P2RX7, P2X7, BCR, CML, |
| | PHL, ALL, GRAF, NF1, VRNF, WSS, NFNS, PTPN11, |
| | PTP2C, SHP2, NS1, BCL2, CCND1, PRAD1, BCL1, |
| | TCRA, GATA1, GF1, ERYF1, NFE1, ABL1, NQO1, |
| | DIA4, NMOR1, NUP214, D9S46E, CAN, CAIN) |
| Inflammation and | AIDS (KIR3DL1, NKAT3, NKB1, AMB11, KIR3DS1, |
| immune related diseases | IFNG, CXCL12, SDF1); Autoimmune lymphoproliferative |
| and | syndrome (TNFRSF6, APT1, FAS, CD95, ALPS1A); |
| disorders | Combined immunodeficiency, (IL2RG, SCIDX1, SCIDX, |
| | IMD4); HIV-1 (CCL5, SCYA5, D17S136E, TCP228), HIV |
| | susceptibility or infection (IL10, CSIF, CMKBR2, CCR2, |
| | CMKBR5, CCCKR5 (CCR5)); Immunodeficiencies |
| | (CD3E, CD3G, AICDA, AID, HIGM2, TNFRSF5, CD40, |
| | UNG, DGU, HIGM4, TNFSF5, CD40LG, HIGM1, IGM, |
| | FOXP3, IPEX, AIID, XPID, PIDX, TNFRSF14B, TACI); |
| | Inflammation (IL-10, IL-1 (IL-1a, IL-1b), IL-13, IL-17 (IL- |
| | 17a (CTLA8), IL-17b, IL-17c, IL- 17d, IL-17f), II-23, |
| | Cx3cr1, ptpn22, TNFa, NOD2/CARD15 for IBD, IL-6, IL- |
| | 12 (IL-12a, IL-12b), CTLA4, Cx3cl1); Severe combined |
| | immunodeficiencies (SCIDs)(JAK3, JAKL, DCLRE1C, |
| | ARTEMIS, SCIDA, RAG1, RAG2, ADA, PTPRC, CD45, |
| | LCA, IL7R, CD3D, T3D, IL2RG, SCIDX1, SCIDX, |
| | IMD4) |
| Metabolic, liver, kidney | Amyloid neuropathy (TTR, PALB); Amyloidosis (APOA1, |
| and protein diseases and | APP, AAA, CVAP, AD1, GSN, FGA, LYZ, TTR, PALB); |
| disorders | Cirrhosis (KRT18, KRT8, CIRH1A, NAIC, TEX292, |
| | KIAA1988); Cystic fibrosis (CFTR, ABCC7, CF, MRP7); |
| | Glycogen storage diseases (SLC2A2, GLUT2, G6PC, |
| | G6PT, G6PT1, GAA, LAMP2, LAMPB, AGL, GDE, |
| | GBE1, GYS2, PYGL, PFKM); Hepatic adenoma, 142330 |
| | (TCF1, HNF1A, MODY3), Hepatic failure, early onset, |
| | and neurologic disorder (SCOD1, SCO1), Hepatic lipase |
| | deficiency (LIPC), Hepatoblastoma, cancer and carcinomas |
| | (CTNNB1, PDGFRL, PDGRL, PRLTS, AXIN1, AXIN, |
| | CTNNB1, TP53, P53, LFS1, IGF2R, MPRI, MET, CASP8, |
| | MCH5; Medullary cystic kidney disease (UMOD, HNFJ, |
| | FJHN, MCKD2, ADMCKD2); Phenylketonuria (PAH, |
| | PKU1, QDPR, DHPR, PTS); Polycystic kidney and hepatic |
| | disease (FCYT, PKHD1, ARPKD, PKD1, PKD2, PKD4, |
| | PKDTS, PRKCSH, G19P1, PCLD, SEC63) |
| Muscular/Skeletal | Becker muscular dystrophy (DMD, BMD, MYF6), |
| diseases and disorders | Duchenne Muscular Dystrophy (DMD, BMD); Emery- |
| | Dreifuss muscular dystrophy (LMNA, LMN1, EMD2, |
| | FPLD, CMD1A, HGPS, LGMD1B, LMNA, LMN1, |
| | EMD2, FPLD, CMD1A); Facioscapulohumeral muscular |
| | dystrophy (FSHMD1A, FSHD1A); Muscular dystrophy |
| | (FKRP, MDC1C, LGMD2I, LAMA2, LAMM, LARGE, |
| | KIAA0609, MDC1D, FCMD, TTID, MYOT, CAPN3, |
| | CANP3, DYSF, LGMD2B, SGCG, LGMD2C, DMDA1, |
| | SCG3, SGCA, ADL, DAG2, LGMD2D, DMDA2, SGCB, |
| | LGMD2E, SGCD, SGD, LGMD2F, CMD1L, TCAP, |
| | LGMD2G, CMD1N, TRIM32, HT2A, LGMD2H, FKRP, |
| | MDC1C, LGMD2I, TTN, CMD1G, TMD, LGMD2J, |
| | POMT1, CAV3, LGMD1C, SEPN1, SELN, RSMD1, |
| | PLEC1, PLTN, EBS1); Osteopetrosis (LRP5, BMND1, |
| | LRP7, LR3, OPPG, VBCH2, CLCN7, CLC7, OPTA2, |
| | OSTM1, GL, TCIRG1, TIRC7, OC116, OPTB1); Muscular |
| | atrophy (VAPB, VAPC, ALS8, SMN1, SMA1, SMA2, |
| | SMA3, SMA4, BSCL2, SPG17, GARS, SMAD1, CMT2D, |
| | HEXB, IGHMBP2, SMUBP2, CATF1, SMARD1) |
| Dermatological diseases | Albinisim (TYR, OCA2, TYRP1, SLC45A2, LYST), |
| and disorders | Ectodermal dysplasias (EDAR, EDARADD, WNT10A), |
| | Ehlers-Danlos syndrome (COL5A1, COL5A2, COL1A1, |
| | COL1A2, COL3A1, TNXB, ADAMTS2, PLOD1, |
| | FKBP14), Ichthyosis-associated disorders (FLG, STS, |
| | TGM1, ALOXE3/ALOX12B, KRT1, KRT10, ABCA12, |
| | KRT2, GJB2, TGM1, ABCA12, CYP4F22, ALOXE3, |
| | CERS3, NSHDL, EBP, MBTPS2, GJB2, SPINK5, |
| | AGHD5, PHYH, PEX7, ALDH3A2, ERCC2, ERCC3, |
| | GFT2H5, GBA), Incontinentia pigmenti (IKBKG, NEMO), |
| | Tuberous sclerosis (TSC1, TSC2), Premature aging |
| | syndromes (POLR3A, PYCR1, LMNA, POLD1, WRN, |
| | DMPK) |
| Neurological and | ALS (SOD1, ALS2, STEX, FUS, TARDBP, VEGF |
| Neuronal diseases and | (VEGF-a, VEGF-b, VEGF-c); Alzheimer disease (APP, |
| disorders | AAA, CVAP, AD1, APOE, AD2, PSEN2, AD4, STM2, |
| | APBB2, FE65L1, NOS3, PLAU, URK, ACE, DCP1, |
| | ACE1, MPO, PACIP1, PAXIP1L, PTIP, A2M, BLMH, |
| | BMH, PSEN1, AD3); Autism (Mecp2, BZRAP1, MDGA2, |
| | Sema5A, Neurexin 1, GLO1, MECP2, RTT, PPMX, |
| | MRX16, MRX79, NLGN3, NLGN4, KIAA1260, |
| | AUTSX2); Fragile X Syndrome (FMR2, FXR1, FXR2, |
| | mGLUR5); Huntington's disease and disease like disorders |
| | (HD, IT15, PRNP, PRIP, JPH3, JP3, HDL2, TBP, SCA17); |
| | Parkinson disease (NR4A2, NURR1, NOT, TINUR, |
| | SNCAIP, TBP, SCA17, SNCA, NACP, PARK1, PARK4, |
| | DJ1, PARK7, LRRK2, PARK8, PINK1, PARK6, UCHL1, |
| | PARK5, SNCA, NACP, PARK1, PARK4, PRKN, PARK2, |
| | PDJ, DBH, NDUFV2); Rett syndrome (MECP2, RTT, |
| | PPMX, MRX16, MRX79, CDKL5, STK9, MECP2, RTT, |
| | PPMX, MRX16, |
| | MRX79, x-Synuclein, DJ-1); Schizophrenia (Neuregulin1 |
| | (Nrg1), Erb4 (receptor for Neuregulin), Complexin1 |
| | (Cplx1), Tph1 Tryptophan hydroxylase, Tph2, Tryptophan |
| | hydroxylase 2, Neurexin 1, GSK3, GSK3a, GSK3b, 5-HTT |
| | (Slc6a4), COMT, DRD (Drd1a), SLC6A3, DAOA, |
| | DTNBP1, Dao (Dao1)); Secretase Related Disorders |
| | (APH-1 (alpha and beta), Presenilin (Psen1), nicastrin, |
| | (Ncstn), PEN-2, Nos1, Parp1, Natl, Nat2); Trinucleotide |
| | Repeat Disorders (HTT (Huntington's Dx), |
| | SBMA/SMAX1/AR (Kennedy's Dx), FXN/X25 |
| | (Friedrich's Ataxia), ATX3 (Machado-Joseph's Dx), |
| | ATXN1 and ATXN2 (spinocerebellar ataxias), DMPK |
| | (myotonic dystrophy), Atrophin-1 and Atn1 (DRPLA Dx), |
| | CBP (Creb-BP - global instability), VLDLR (Alzheimer's), |
| | Atxn7, Atxn10) |
| Ocular diseases and | Age-related macular degeneration (Abcr, Ccl2, Cc2, cp |
| disorders | (ceruloplasmin), Timp3, cathepsinD, Vldlr, Ccr2); Cataract |
| | (CRYAA, CRYA1, CRYBB2, CRYB2, PITX3, BFSP2, |
| | CP49, CP47, CRYAA, CRYA1, PAX6, AN2, MGDA, |
| | CRYBA1, CRYB1, CRYGC, CRYG3, CCL, LIM2, MP19, |
| | CRYGD, CRYG4, BFSP2, CP49, CP47, HSF4, CTM, |
| | HSF4, CTM, MIP, AQP0, CRYAB, CRYA2, CTPP2, |
| | CRYBB1, CRYGD, CRYG4, CRYBB2, CRYB2, CRYGC, |
| | CRYG3, CCL, CRYAA, CRYA1, GJA8, CX50, CAE1, |
| | GJA3, CX46, CZP3, CAE3, CCM1, CAM, KRIT1); |
| | Corneal clouding and dystrophy (APOA1, TGFBI, CSD2, |
| | CDGG1, CSD, BIGH3, CDG2, TACSTD2, TROP2, M1S1, |
| | VSX1, RINX, PPCD, PPD, KTCN, COL8A2, FECD, |
| | PPCD2, PIP5K3, CFD); Cornea plana congenital (KERA, |
| | CNA2); Glaucoma (MYOC, TIGR, GLC1A, JOAG, |
| | GPOA, OPTN, GLC1E, FIP2, HYPL, NRP, CYP1B1, |
| | GLC3A, OPA1, NTG, NPG, CYP1B1, GLC3A); Leber |
| | congenital |
| | amaurosis (CRB1, RP12, CRX, CORD2, CRD, RPGRIP1, |
| | LCA6, CORD9, RPE65, RP20, AIPL1, LCA4, GUCY2D, |
| | GUC2D, LCA1, CORD6, RDH12, LCA3); Macular |
| | dystrophy (ELOVL4, ADMD, STGD2, STGD3, RDS, |
| | RP7, PRPH2, PRPH, AVMD, AOFMD, VMD2) |

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease with the targeting sequence serving as the targeting portion of the CRISPR complex. When the molecule having the targeting sequence is present contemporaneously with the CRISPR molecule the RNA molecule is capable of targeting the CRISPR nuclease to the specific target sequence. Each possibility represents a separate embodiment. An RNA molecule can be custom designed to target any desired sequence.

The term “targets” as used herein, refers to a targeting sequence or targeting molecule's preferential hybridization to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

As used herein the term “wild-type” is a term of the art understood by skilled persons and means the typical form of an organism, strain, gene or characteristic as it occurs in nature as distinguished from mutant or variant forms. Accordingly, as used herein, where a sequence of amino acids or nucleotides refers to a wild-type sequence, a variant refers to variant of that sequence, e.g., comprising substitutions, deletions, insertions. In embodiments of the present invention, an engineered CRISPR nuclease is a variant CRISPR nuclease comprising at least one amino acid modification (e.g., substitution, deletion, and/or insertion), also referred to as a “mutation,” compared to the wild-type OMNI-50 nuclease of SEQ ID NO: 1.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

The terms “mutant” or “variant” are used interchangeably and indicate a molecule that is non-naturally occurring or engineered.

As used herein the term “amino acid” includes natural and/or unnatural or synthetic amino acids, including glycine and both the D- or L-, optical isomers, and amino acid analogs and peptidomimetics.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

As used herein, the term “modified cells” refers to cells in which a double strand break is affected by a complex of an RNA molecule and the CRISPR nuclease variant as a result of hybridization with the target sequence, i.e. on-target hybridization. The term “modified cells” may further encompass cells in which a repair or correction of a mutation was affected following the double strand break induced by the variant. The modified cell may be any type of cell e.g., eukaryotic or prokaryotic, in any environment e.g., isolated or not, maintained in culture, in vitro, ex vivo, in vivo or in planta.

This invention provides a modified cell or cells obtained by use of any of the variants or methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cell (HSC), or any cell suitable for an allogenic cell transplant or autologous cell transplant. The variants and methods described herein may also be utilized to generate chimeric antigen receptor T (CAR-T) cells.

This invention also provides a composition comprising these modified cells and a pharmaceutically acceptable carrier. Also provided is an in vitro or ex vivo method of preparing this, comprising mixing the cells with the pharmaceutically acceptable carrier.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity.

The terms “protospacer adjacent motif” or “PAM” as used herein refers to a nucleotide sequence of a target DNA located in proximity to the targeted DNA sequence and recognized by the CRISPR nuclease. The PAM sequence may differ depending on the nuclease identity. For example, wild-type Streptococcus pyogenes Cas9 recognizes a “NGG” PAM sequence. A skilled artisan will appreciate that single-guide RNA molecules or crRNA:tracrRNA complexes capable of complexing with a CRISPR nuclease such as to associate with a target genomic DNA sequence of interest next to a protospacer adjacent motif (PAM). The nuclease then mediates cleavage of target DNA to create a double-stranded break within the protospacer.

As used herein, a sequence or molecule has an X % “sequence identity” to another sequence or molecule if X % of bases or amino acids between the sequences of molecules are the same and in the same relative position. For example, a first nucleotide sequence having at least a 95% sequence identity with a second nucleotide sequence will have at least 95% of bases, in the same relative position, identical with the other sequence.

The terms “nuclear localization sequence” and “NLS” are used interchangeably to indicate an amino acid sequence/peptide that directs the transport of a protein with which it is associated from the cytoplasm of a cell across the nuclear envelope barrier. The term “NLS” is intended to encompass not only the nuclear localization sequence of a particular peptide, but also derivatives thereof that are capable of directing translocation of a cytoplasmic polypeptide across the nuclear envelope barrier. NLSs are capable of directing nuclear translocation of a polypeptide when attached to the N-terminus, the C-terminus, or both the N- and C-termini of the polypeptide. In addition, a polypeptide having an NLS coupled by its N- or C-terminus to amino acid side chains located randomly along the amino acid sequence of the polypeptide will be translocated. Typically, an NLS consists of one or more short sequences of positively charged lysines or arginines exposed on the protein surface, but other types of NLS are known. Non-limiting examples of NLSs include an NLS sequence derived from: the SV40 virus large T-antigen, nucleoplasmin, c-myc, the hRNPAl M9 NLS, the IBB domain from importin-alpha, myoma T protein, human p53, mouse c-ab1 IV, influenza vims NS1, Hepatitis virus delta antigen, mouse Mx1 protein, human poly(ADP-ribose) polymerase, and the steroid hormone receptors (human) glucocorticoid.

The term “CRISPR system” refers to a CRISPR endonuclease system that includes a CRISPR nuclease protein, such as the mutants or variants described herein, and a suitable guide RNA molecule or guide RNA complex, e.g. a single-guide RNA or a crRNA:tracrRNA complex, for targeting the CRISPR nuclease protein to a desired target DNA sequence based on complementarity between a portion of the guide RNA molecule or guide RNA complex and the target DNA sequence. The term “wild-type CRISPR endonuclease system” refers to a CRISPR endonuclease system that includes wild-type CRISPR protein and a suitable guide RNA molecule or guide RNA complex, e.g. a single-guide RNA or a crRNA:tracrRNA complex, for targeting the wild-type CRISPR nuclease protein to a desired target DNA sequence based on complementarity between a portion of the guide RNA molecule or guide RNA complex and the target DNA sequence.

In the context of the invention, “maintained on-target editing activity” refers to the ability of an OMNI-50 variant to target a DNA target site that is targeted by a guide RNA molecule associated with, and thereby programming, the OMNI-50 variant. In some embodiments, the OMNI-50 variant maintains on-target editing activity of a DNA target at a percent editing level greater than or equal to the percent editing level of a wild-type OMNI-50 nuclease for the DNA target. In some embodiments, the OMNI-50 variant maintains on-target editing activity of a DNA target of at least 100%, 95%, 90%, 80%, 70%, 60%, 50%, 40%, or 30% the level of percent editing of a wild-type OMNI-50 nuclease for the DNA target.

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques are thoroughly explained in the literature. See, for example, Sambrook et al., “Molecular Cloning: A laboratory Manual” (1989); Ausubel, R. M. (Ed.), “Current Protocols in Molecular Biology” Volumes I-III (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (Eds.), “Genome Analysis: A Laboratory Manual Series”, Vols. 1-4, Cold Spring Harbor Laboratory Press, New York (1998); Methodologies as set forth in U.S. Pat. Nos. 4,666,828; 4,683,202; 4,801,531; 5,192,659 and 5,272,057; Cellis, J. E. (Ed.), “Cell Biology: A Laboratory Handbook”, Volumes I-III (1994); Freshney, “Culture of Animal Cells—A Manual of Basic Technique” Third Edition, Wiley-Liss, N. Y. (1994); Coligan J. E. (Ed.), “Current Protocols in Immunology” Volumes I-III (1994); Stites et al. (Eds.), “Basic and Clinical Immunology” (8th Edition), Appleton & Lange, Norwalk, CT (1994); Mishell and Shiigi (Eds.), “Strategies for Protein Purification and Characterization—A Laboratory Course Manual” CSHL Press (1996); Clokie and Kropinski (Eds.), “Bacteriophage Methods and Protocols”, Volume 1: Isolation, Characterization, and Interactions (2009), all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLES

### Example 1: General Approach

In order to select OMNI-50 nuclease variants with improved functionality, such as improved activity improved fidelity and improved discrimination, amino acid substitutions are introduced into the open reading frame of the wild-type OMNI-50 sequence (SEQ ID NO: 1).

To evaluate functionality of the OMNI-50 variants, the activity of OMNI-50 nuclease variants are compared to activity of wild-type OMNI-50 nuclease. Further, guides designed for discriminating between SNP positions are used for testing OMNI-50 nuclease variant activity and allele-specific editing.

### Example 2: OMNI-50 CRISPR Nuclease Variants

### OMNI-50 CRISPR Nuclease Variant Library

The open reading frame of the wild-type OMNI-50 CRIPSR nuclease was codon optimized for human cell line expression and cloned into a dual expression plasmid (pShuttle) that enables both bacterial and mammalian expression using a T7 or a CMV promoter, respectively. A full gene library with combinatorial random mutations along the full-length OMNI-50 open reading frame (ORF) was constructed using incorporated oligos with an NNK degenerate codon at each position in the OMNI-50 sequence.

### Bacterial-Based Positive Selection Systems

In order to isolate OMNI-50 variants with enhanced activity, a positive selection bacterial system was designed. In this system, a positive selection plasmid was electroporated into Escherichia coli strain BW25141 (lDE3) to create the positive selection bacterial strain. The positive selection plasmid contains a T7-expressed single-guide RNA (sgRNA) and an embedded on-target site. The sequence of the target site, which is a sequence located upstream to the human ELANE gene, and the spacer and scaffold sequences of the guide RNA molecule, are listed in Table 5.

The positive selection plasmid also contains a chloramphenicol resistance cassette and expresses the E. coli toxin gene CcdB under the control of an araBAD promoter. Accordingly, upon electroporating the OMNI-50 library pool, which contains the OMNI-50 variants, into the positive selection bacterial strain, only bacterial colonies that express an active OMNI-50 variant which cleaves the positive selection plasmid, thus neutralizing the toxin, are able to survive on selective plates containing arabinose.

Following a 10-minute post-electroporation recovery period in TB media, transformed bacteria are plated on selective TB plates containing carbenicillin and 15 mM arabinose and incubated overnight at 37° C. The next morning the surviving pool is collected, plasmids are isolated, and re-transformed into the positive selection bacterial strain for another round of selection. The first mutagenesis generation included three rounds of positive selection followed by second generation of mutagenesis and six more rounds of positive selections. After the final round, single bacterial colonies were randomly picked and fully sequenced. Unique variants were cloned into pET9 vector for protein production as RNP.

### High Grade Protein Expression and Purification

WT OMNI-50 and its variants were cultured in autoinduction TB media at 37° C., 250 rpm till OD₆₀₀ 1 is reached, then temperature shifted to 20° C. for 17-20 hours. Cells were harvested by centrifugation at 6000×g for 20 min and stored at −80° C. Cells were lysed using chemical lysis followed by centrifugation. Cleared lysate was purified on Ni-NTA resin. Ni-NTA elution fraction was purified on CEX (S03 Fractogel) resin followed by SEC purification on Superdex 200 Increase 10/300 GL on AKTA Pure (GE Healthcare Life Sciences). Fractions containing OMNI-50 variants were pooled and concentrated to 20 mg/ml, filtered 0.22 um and flash-frozen in liquid nitrogen and stored at −80° C.

### HTP Protein Expression and Purification

WT OMNI-50 and its variants were cultured in autoinduction TB media at 37° C., 350 rpm for 3.5 hours, and shifted to 18° C. for 17-20 hours. Cells were harvested by centrifugation at 4000×g and stored at −80° C. OMNI-50 variants and WT cell pellets were thawed and incubated in lysis buffer for 30 minutes. Crude lysate was clarified by centrifugation at 4000×g for 1 hour, 4° C. Cleared protein lysates were incubated with Sepharose 6 Ni-NTA resin (Cytiva). Protein-bound Ni-NTA resin was loaded on a 96 Filter well plate and washed with buffer (HEPES 20 mM, NaCl 0.6 M, Imidazole 60 mM) to remove contaminants. Protein was eluted from the resin with high concentrations of Imidazole buffer (HEPES 20 mM, NaCl 0.6 M, Imidazole 0.4 M). Eluted proteins were desalted using 96 filter plates containing 1800 μl of G-25 Sephadex resin equilibrated with storage buffer. Desalted proteins were then concentrated (Amicon-ultra 0.5 ml 50 kDa, Millipore) and sterile filtered (Ultrafree-MC 0.22 μm PVDF filters). Purified variants were then stored at −80° C. and analyzed for concentration, purity, and in vitro activity before transfection to cells.

### Screening in Mammalian Cells and Cleavage Analysis

RNP mixture was prepared by mixing 124 μmol of sgRNA listed in Table 5 and 105 pmol of nuclease for 10 minutes at room temp. HSCs homozygous or heterozygous to ELANE_g58 target site, or SH-SY5Y cells homozygous to hSARM1_g92, were centrifugated at room temperature, at 300 g for 5 minutes and washed with PBS. The pellet was re-suspended in an appropriate volume of Lonza Nucleofection electroporation solution and transferred to the RNP mixture. Electroporation was performed in triplicates or duplicates using 4D-Nucleofector device (Lonza Bioscience). Pre-warmed medium was added to the cuvette immediately after electroporation. Cells were incubated for 3 days (37° C., 5% C02). At 72h, cells were harvested, and their genomic DNA content was used as template in a PCR reaction, amplifying the corresponding cleavage targets and off targets (if known). Amplicons were subjected to NGS and the resulting sequences were then used to calculate the percentage of editing events in each target site. Short insertions or deletions (indels) around the cut site are the typical outcome of DNA repair following nuclease induced DNA cleavage. The calculation of editing percentage was therefore deduced from the fraction of indels containing sequences within each amplicon.

### Protein Residual Activity Determination

To determine thermal stability, proteins were diluted to 100 nM and incubated at 25° C. and 44° C. for 10 minutes. After incubation, 150 nM gRNA was added and incubated at 25° C. for 10 minutes. Next, 100 nM DNA was added to the RNP and cleavage activity was measured. Percent residual activity was calculated by dividing the activity level at 44° C. by the activity level at 25° C.

### Results

### Increased Activity and Fidelity of Two OMNI-50 Variants at a Selected Test Target Site

Two leading variants, OMNI-50 V6552 and V6172 (Table 2), were isolated from the bacterial selections and showed significant increased activity relative to the WT OMNI-50 nuclease.

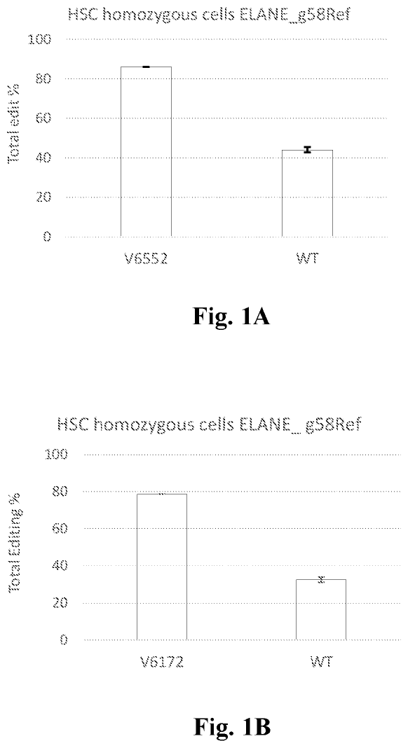

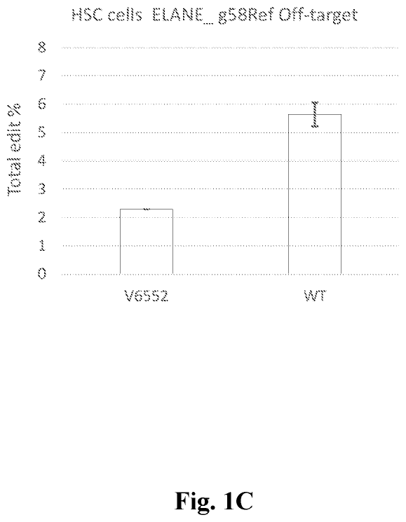

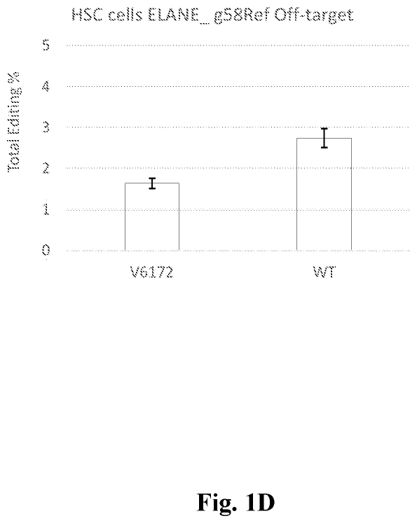

In homozygous HSCs for ELANE_g58Ref, both variants showed a significant increase in activity on the human target site that was used in the selection system relative to the WT RNP (FIGS. 1A and 1B). In addition to the high activity of those variants, they also displayed increased fidelity as observed by reduction in off-target editing levels of the g58Ref target (FIGS. 1C and 1D)

### OMNI-50 Variants Show Broad Increased Activity on Different Sites

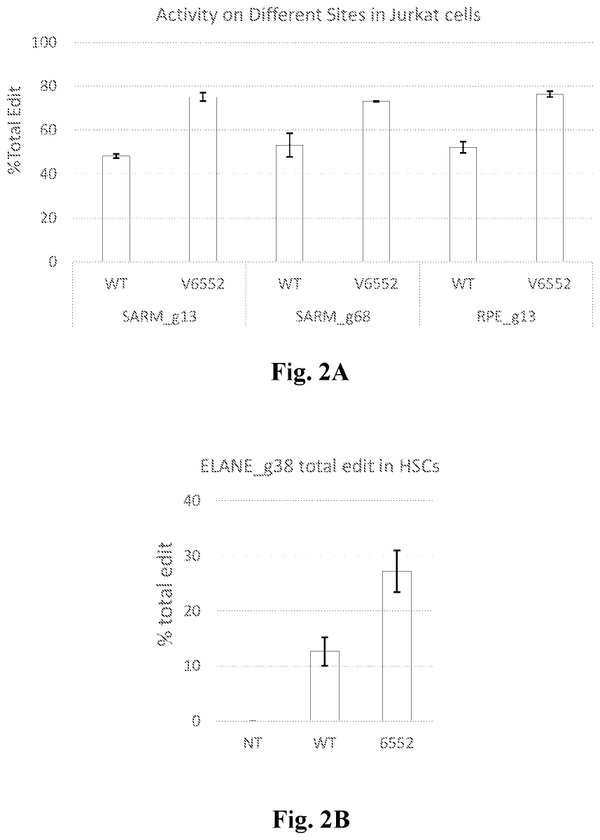

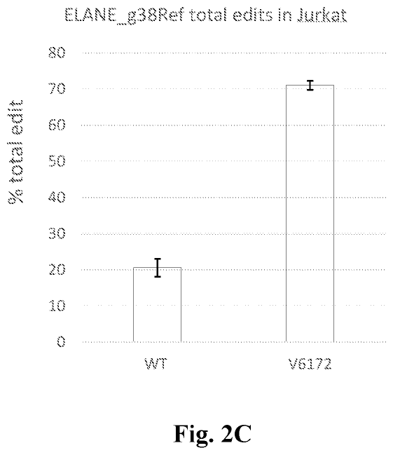

The two leading variants also show significant increased activity relative to the WT RNP on additional sites when tested in mammalian cells (FIGS. 2A-2C, Table 5). When tested in Jurkat cells, V6552 showed higher activity than WT OMNI-50 on the SARM_g13, SARM_g68, and RPE_g13 target sites. V6552 also showed higher activity than WT OMNI-50 on ELANE_g38Ref when tested in an HSC cell line (FIG. 2B). Additionally, V6172 shows increased activity on ELANE_g38Ref when tested in Jurkat cells (FIG. 2C).

### OMNI-50 Variants Show Increased Specificity

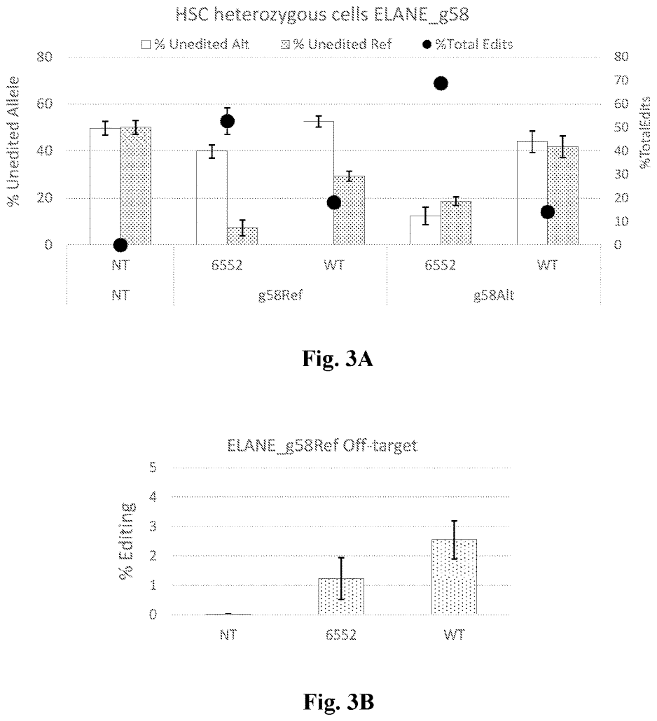

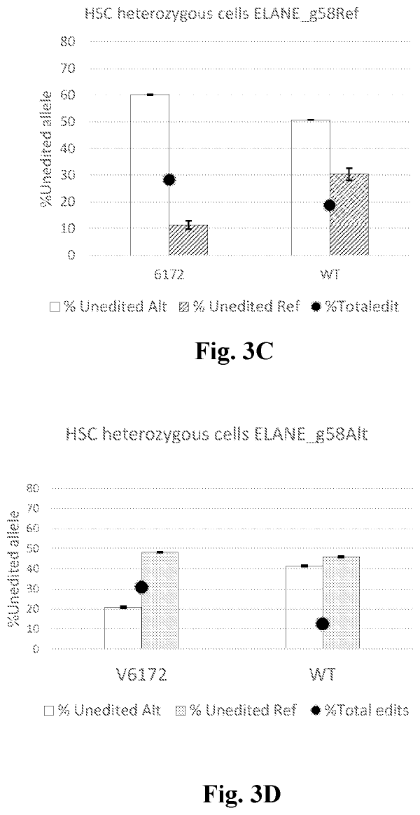

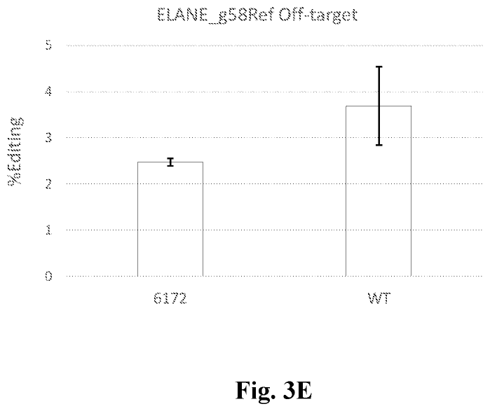

Discrimination between two heterozygous alleles requires high specificity from the nuclease than fidelity, as the undesired DNA site may differ by only a single nucleotide from the target DNA site. When tested in HSCs that are heterozygous for an ELANE SNP using the selected g58Ref, both variants showed an increased discrimination profile compared to WT OMNI-50 as demonstrated by the higher fraction of unedited Alt allele compared to the targeted Ref allele (FIGS. 3A and 3C). However, when treated with the Alt guide RNA, only V6172 showed full discrimination, indicating that it has the higher specificity of the two. Nevertheless, both variants retain higher fidelity levels than WT, as indicated by lower off-target editing (FIGS. 3B and 3E).

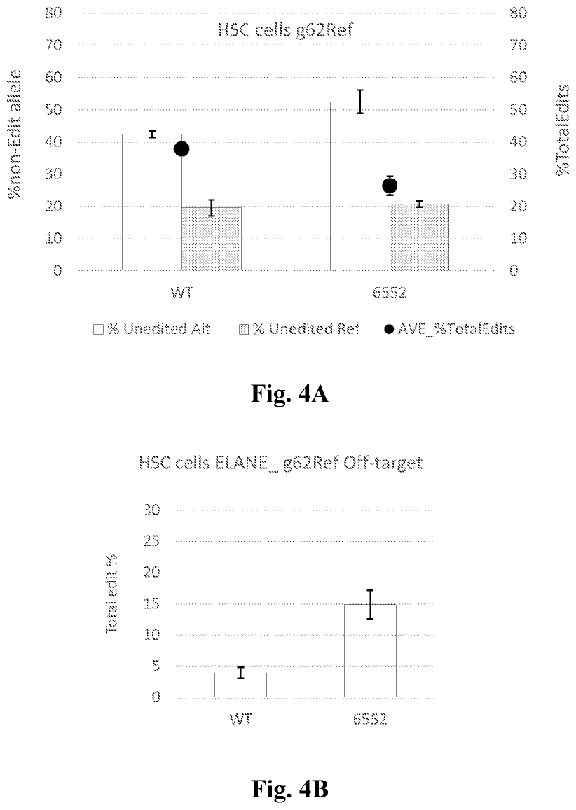

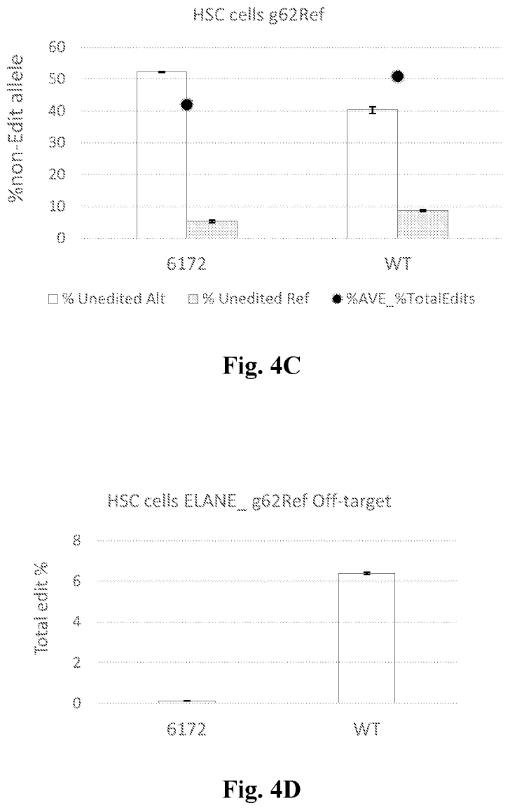

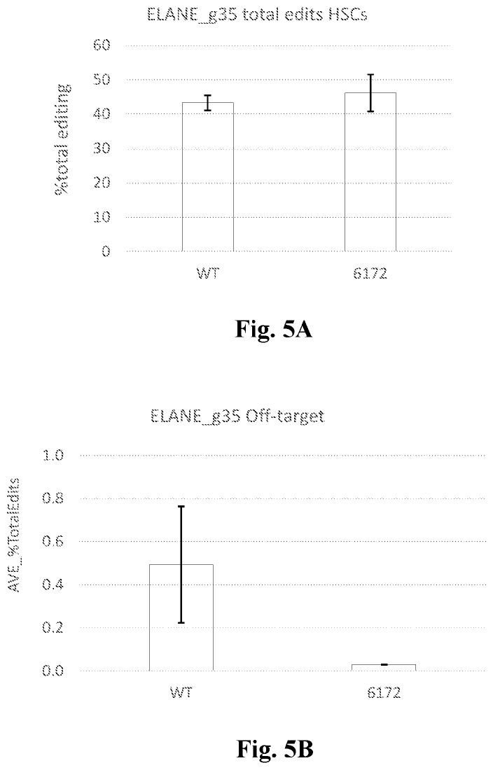

We further tested the ability of the V6172 and V6552 variants to discriminate between alleles on another ELANE SNP (Target 62, Table 5). As can be seen in FIGS. 4A-4D, when cells are treated with g62Ref the WT OMNI-50 protein cleaves mainly the Ref allele but shows 10% non-specific editing on the Alt allele. However, V6552 showed specific editing on the Ref targeted allele without non-specific edits on the Alt allele (FIG. 4A). Again, V6172 shows a higher discrimination profile by almost full cleavage of the targeted Ref allele without touching the Alt allele (FIG. 4C). This specificity is also manifested by a reduction in the off-targets of g62Ref for this variant, which is not the case for V6552. This indicates that in this case specificity may be sequence dependent (FIG. 4D). V6172 is also active on g35 and has decreased off-targets compared to WT OMNI-50 (0.03% and 0.49%, respectively—FIGS. 5A-5B).

TABLE 2

| | | Substitu- | | | | | | | | | | | | | |
|---|---|---|---|---|---|---|---|---|---|---|---|---|---|---|---|
| | | tions | | | | | | | | | | | | | |
| | | Relative | | | | | | | | | | | | | |
| SEQ | | to Wild- | | | | | | | | | | | | | |
| ID | Variant | type | Numbered position in amino acid sequence and corresponding residue (“—” denotes no change relative to WT OMNI-50) | | | | | | | | | | | | |
| NO. | Name | OMNI-50 | 252 | 281 | 300 | 302 | 368 | 520 | 614 | 698 | 779 | 836 | 939 | 1100 | 1339 |
| 1 | Wild-type | — | D | D | N | L | N | E | G | N | S | E | T | L | S |
| | OMNI-50 | | | | | | | | | | | | | | |
| 2 | V6552 | D252Y | Y | V | — | N | S | — | — | — | — | — | — | F | R |
| | | D281V | | | | | | | | | | | | | |
| | | L302N | | | | | | | | | | | | | |
| | | N368S | | | | | | | | | | | | | |
| | | L1100F | | | | | | | | | | | | | |
| | | S1339R | | | | | | | | | | | | | |
| 16 | V6172 | N300A | — | — | A | — | — | — | R | L | — | F | L | F | — |
| | | G614R | | | | | | | | | | | | | |
| | | N698L | | | | | | | | | | | | | |
| | | E836F | | | | | | | | | | | | | |
| | | T939L | | | | | | | | | | | | | |
| | | L1100F | | | | | | | | | | | | | |
| 28 | V7765 | N300A | — | — | A | — | — | — | R | L | P | F | L | F | R |
| | | G614R | | | | | | | | | | | | | |
| | | N698L | | | | | | | | | | | | | |
| | | S779P | | | | | | | | | | | | | |
| | | E836F | | | | | | | | | | | | | |
| | | T939L | | | | | | | | | | | | | |
| | | L1100F | | | | | | | | | | | | | |
| | | S1339R | | | | | | | | | | | | | |
| 29 | V7261 | S779P | — | — | — | — | — | — | — | — | P | — | — | — | — |
| 30 | V7281 | D252Y | Y | V | — | N | S | — | — | — | P | — | — | F | R |
| | | D281V | | | | | | | | | | | | | |
| | | L302N | | | | | | | | | | | | | |
| | | N368S | | | | | | | | | | | | | |
| | | S779P | | | | | | | | | | | | | |
| | | L1100F | | | | | | | | | | | | | |
| | | S1339R | | | | | | | | | | | | | |

TABLE 3

| | | Substitutions | | | | | | |
|---|---|---|---|---|---|---|---|---|
| SEQ | | Relative to | | | | | | |
| ID | Variant | Wild-type | Numbered position in amino acid sequence and corresponding residue (“—” denotes no change relative to WT OMNI-50) | | | | | |
| NO. | Name | OMNI-50 | 252 | 281 | 302 | 368 | 1100 | 1339 |
| 1 | Wild- | — | — | — | — | — | — | — |
| | type | | | | | | | |
| | OMNI- | | | | | | | |
| | 50 | | | | | | | |
| 2 | V6552 | D252Y | Y | V | N | S | F | R |
| | | D281V | | | | | | |
| | | L302N | | | | | | |
| | | N368S | | | | | | |
| | | L1100F | | | | | | |
| | | S1339R | | | | | | |
| 3 | V7101 | S1339R | — | — | — | — | — | R |
| 4 | V7896 | L1100F | — | — | — | — | F | — |
| 5 | V7274 | D252Y | Y | — | — | — | — | — |
| 6 | V7275 | D281V | — | V | — | — | — | — |
| 7 | V7276 | L302N | — | — | N | — | — | — |
| 8 | V7277 | N368S | — | — | — | S | — | — |
| 9 | V7253 | D281V | — | V | N | S | F | R |
| | | L302N | | | | | | |
| | | N368S | | | | | | |
| | | L1100F | | | | | | |
| | | S1339R | | | | | | |
| 10 | V7254 | D252Y | Y | — | N | S | F | R |
| | | L302N | | | | | | |
| | | N368S | | | | | | |
| | | L1100F | | | | | | |
| | | S1339R | | | | | | |
| 11 | V7255 | D252Y | Y | V | — | S | F | R |
| | | D281V | | | | | | |
| | | N368S, | | | | | | |
| | | L1100F | | | | | | |
| | | S1339R | | | | | | |
| 12 | V7256 | D252Y | Y | V | N | S | F | — |
| | | D281V | | | | | | |
| | | L302N | | | | | | |
| | | N368S | | | | | | |
| | | L1100F | | | | | | |
| 13 | V7257 | D252Y | Y | V | N | S | — | R |
| | | D281V | | | | | | |
| | | L302N | | | | | | |
| | | N368S | | | | | | |
| | | L1100F | | | | | | |
| | | S1339R | | | | | | |
| 14 | V7134 | D252Y | Y | V | N | — | F | R |
| | | D281V | | | | | | |
| | | L302N | | | | | | |
| | | L1100F | | | | | | |
| | | S1339R | | | | | | |
| 15 | V7492 | L1100F | — | — | — | — | F | R |
| | | S1339R | | | | | | |

TABLE 4

| | | Substitutions | | | | | | |
|---|---|---|---|---|---|---|---|---|
| SEQ | | Relative to | Numbered position in amino acid sequence and corresponding residue | | | | | |
| ID | Variant | Wild-type | (“—” denotes no change relative to WT OMNI-50) | | | | | |
| NO. | Name | OMNI-50 | 300 | 614 | 698 | 836 | 939 | 1100 |
| 1 | Wild- | — | — | — | — | — | — | — |
| | type | | | | | | | |
| | OMNI- | | | | | | | |
| | 50 | | | | | | | |
| 16 | V6172 | N300A | A | R | L | F | L | F |
| | | G614R | | | | | | |
| | | N698L | | | | | | |
| | | E836F | | | | | | |
| | | T939L | | | | | | |
| | | L1100F | | | | | | |
| 17 | V7138 | G614R | — | R | L | F | L | F |
| | | N698L | | | | | | |
| | | E836F | | | | | | |
| | | T939L | | | | | | |
| | | L1100F | | | | | | |
| 18 | V7139 | N300A | A | — | L | F | L | F |
| | | N698L | | | | | | |
| | | E836F | | | | | | |
| | | T939L | | | | | | |
| | | L1100F | | | | | | |
| 19 | V7140 | N300A | A | R | — | F | L | F |
| | | G614R | | | | | | |
| | | E836F | | | | | | |
| | | T939L | | | | | | |
| | | L1100F | | | | | | |
| 20 | V7141 | N300A | A | R | L | — | L | F |
| | | G614R | | | | | | |
| | | N698L | | | | | | |
| | | T939L | | | | | | |
| | | L1100F | | | | | | |
| 21 | V7142 | N300A | A | R | L | F | — | F |
| | | G614R | | | | | | |
| | | N698L | | | | | | |
| | | E836F | | | | | | |
| | | L1100F | | | | | | |
| 22 | V7143 | N300A | A | R | L | F | L | — |
| | | G614R | | | | | | |
| | | N698L | | | | | | |
| | | E836F | | | | | | |
| | | T939L | | | | | | |
| 23 | V7239 | N300A | A | — | — | — | — | — |
| 24 | V7240 | G614R | — | R | — | — | — | — |
| 25 | V7241 | N698L | — | — | L | — | — | — |
| 26 | V7242 | E836F | — | — | — | F | — | — |
| 27 | V7243 | T939L | — | — | — | — | L | — |
| 4 | V7896 | L1100F | — | — | — | — | — | F |

### Contribution of Each of the Mutations of Variant 6552 to the Activity and Specificity of the Nuclease

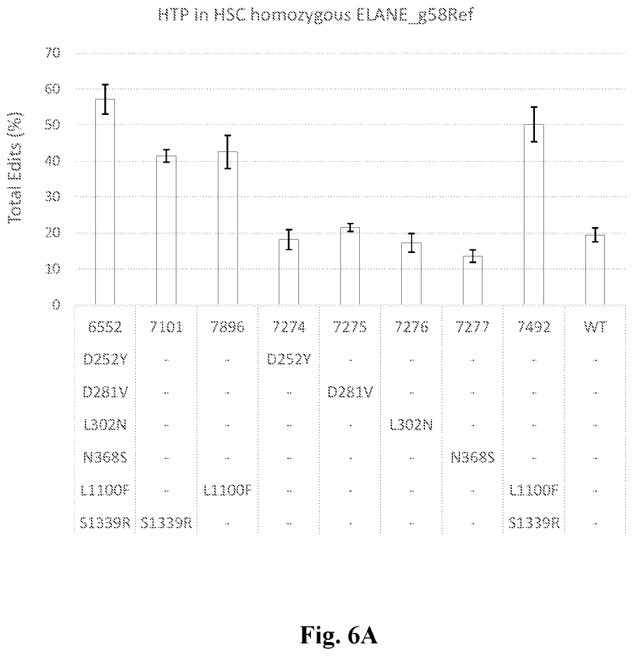

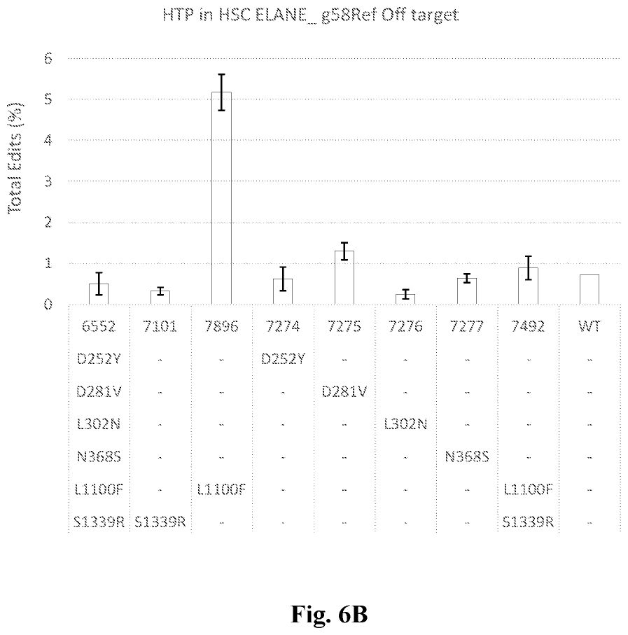

OMNI-50 V6552 contains six (6) mutations: D252Y, D281V, L302N, N686S, L1100F and S1339R (Table 2). To better understand the effect and contribution of each mutation to OMNI-50 V6552 activity, we expressed and purified in HTP format all of the single V6552 variants (i.e. each variant had one of the six V6552 mutations) and tested their activity and fidelity as RNPs in homozygous HSC cells for the target site ELANE_g58Ref, as well as with the off-target site for this guide sequence (Table 3, FIGS. 6A-6B). The mutations L1100F and S1339R are the mutations that contribute the most for the increased activity, as indicated by the variants containing their single mutations (V7101 for the S1339R mutation, and V7896 for the L1100F mutation). The combination of these two mutations (V7492) shows even higher increased activity compared to each of the mutations individually. While L1100F also increases the off-target activity (V7896, FIG. 6B), S1339R shows reduced levels of editing on the g58Ref off-target compared to WT (V7101, FIG. 6B), indicating that this mutation may contribute to the increased specificity of V6552.

Although the other four (4) single mutations show reduced activity on the WT background (V7274, V7275, V7276 and V7277—FIG. 6A), they do not affect the activity on the full variant V6552, suggesting a possible contribution to specificity.

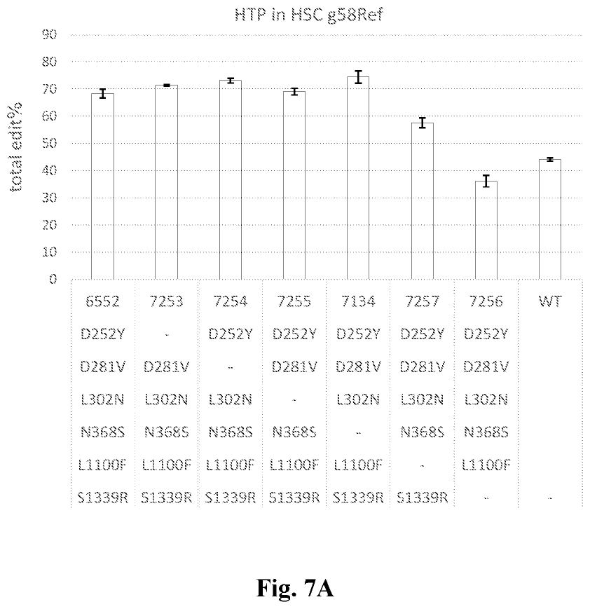

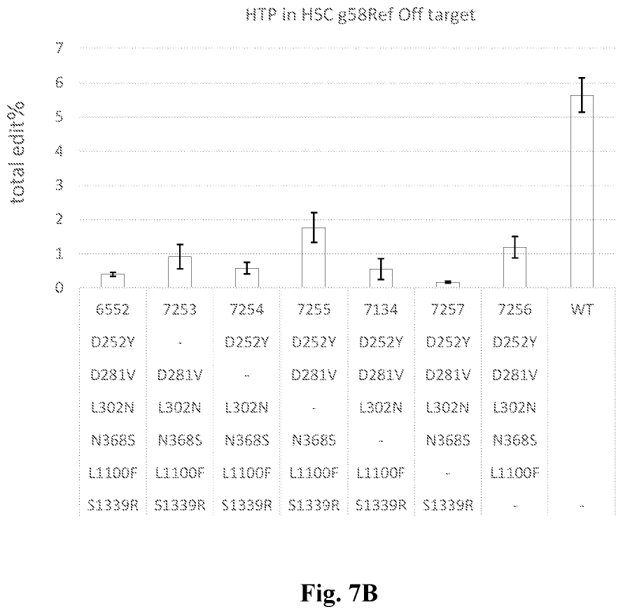

We also tested how reverting each of the single mutations back to a WT substitution relative to V6552 affects activity and fidelity. All variants were expressed and purified in HTP format, and their activity and fidelity as RNPs in homozygous HSC cells for the target site ELANE_g58Ref, along with the off-target site for this guide, was tested (Table 3, FIGS. 7A-7B). Removing L1100F (V7257) or S1339R (V7256) reduces the activity relative to V6552, again highlighting the importance of these mutations to variant activity. When removing the mutations D252Y (V7253) and L302N (V7255), there is increased editing in the off-target site relative to V6552, showing the contribution of these mutations to the specificity of V6552.

### Contribution of Each of the Mutations of Variant 6172 to the Activity and Specificity of the Nuclease

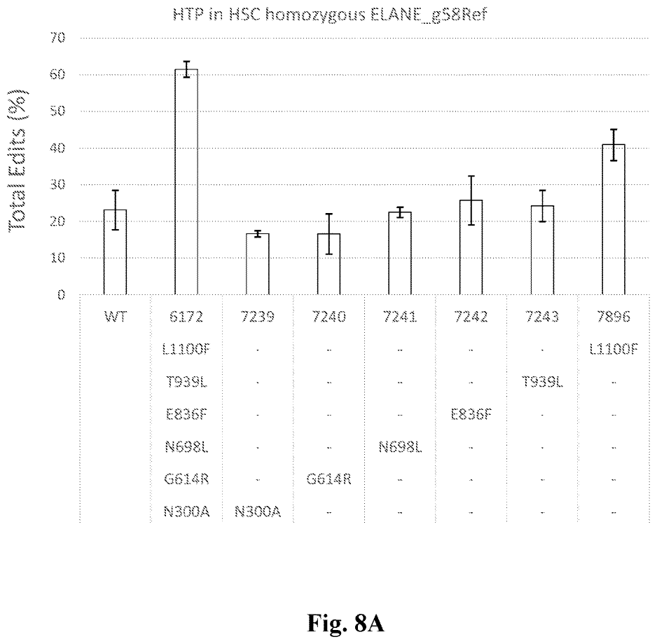

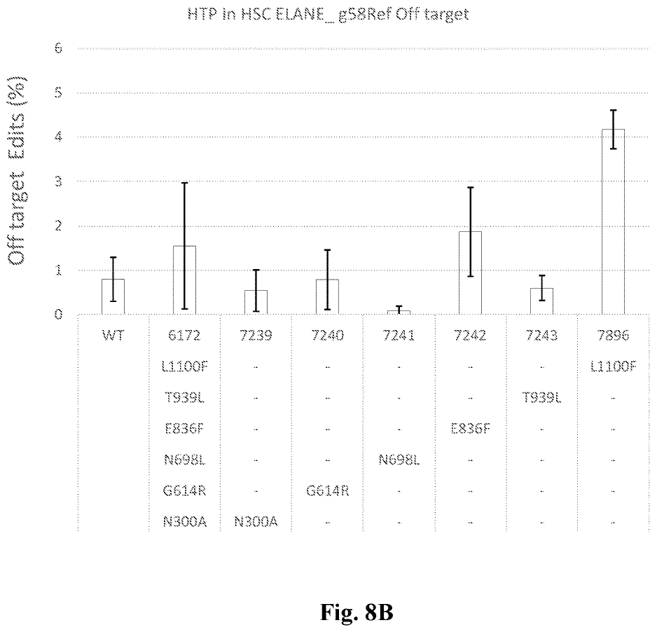

OMNI-50 V6172 contains six (6) mutations: N300A, G614R, N698L, E836F, T939L and L1100F (Table 2). To better understand the effect and contribution of each mutation to OMNI-50 V6172 activity, we expressed and purified in HTP format all of the single V6172 variants (i.e. each variant had one of the six V6552 mutations) and tested their activity and fidelity as RNPs in homozygous HSC cells for the target site ELANE_g58Ref, along with the off-target site for this guide (FIGS. 8A-8B). Here, L1100F (V7896) appears as the mutation that contributes the most to the activity relative to WT (FIG. 8A). However, since V6172 has higher activity than L1100F on its own, any on the other mutations of V6172 may contribute an additional increase in activity. The single mutants N300A (V7239), N698L (V7241), and T939L (V7243) show reduction of off-target levels, indicating that they play a role in fidelity levels (FIG. 8B).

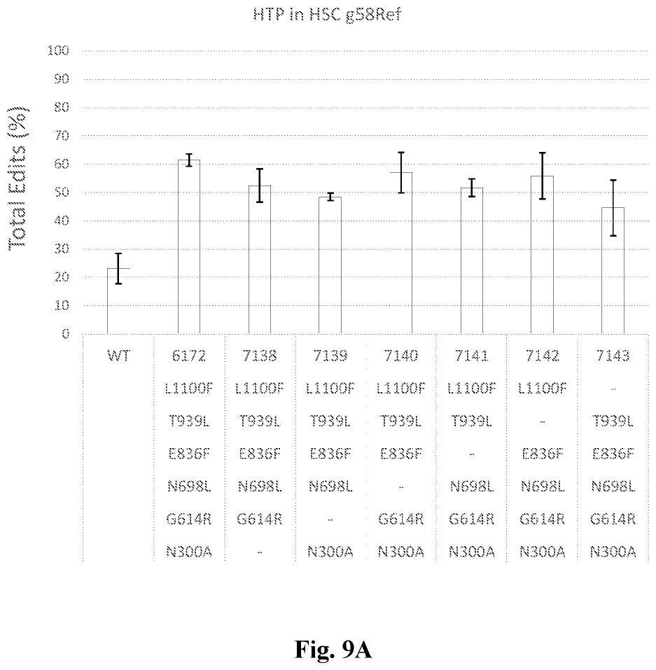

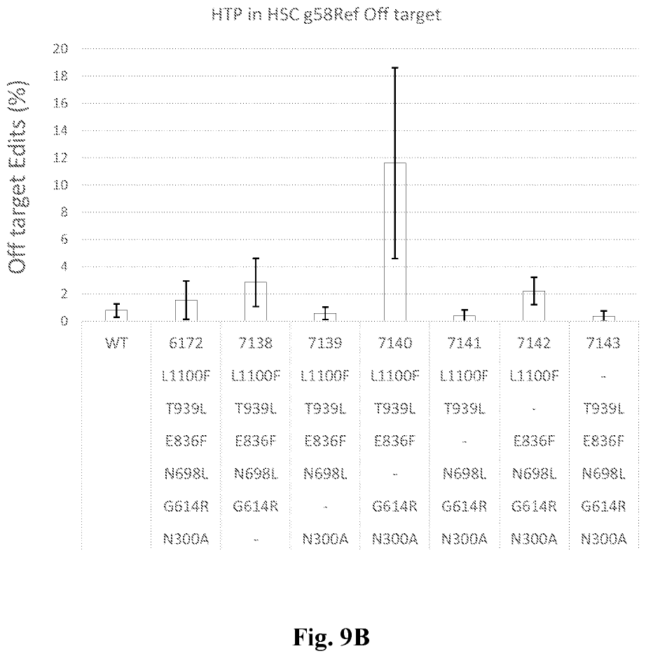

We also tested how reverting each of the single mutations back to a WT substitution relative to V6712 affects activity and fidelity. All variants were expressed and purified in HTP format, and their activity and fidelity as RNPs in homozygous HSC cells for the target site ELANE_g58Ref, along with the off-target site for this guide, was tested. (Table 4, FIGS. 9A-9B). On g58Ref, removing L1100F (V7143) reduces the activity relative to V6172 (FIG. 9A). However, the reduction was not to WT levels, indicating the contribution of other mutations to activity. Removing one of the mutations, N300A (V7138), T939L (V7142), or N698L (V7140), in particular, increases off target compared to V6172, showing their contribution to specificity (FIG. 9B).

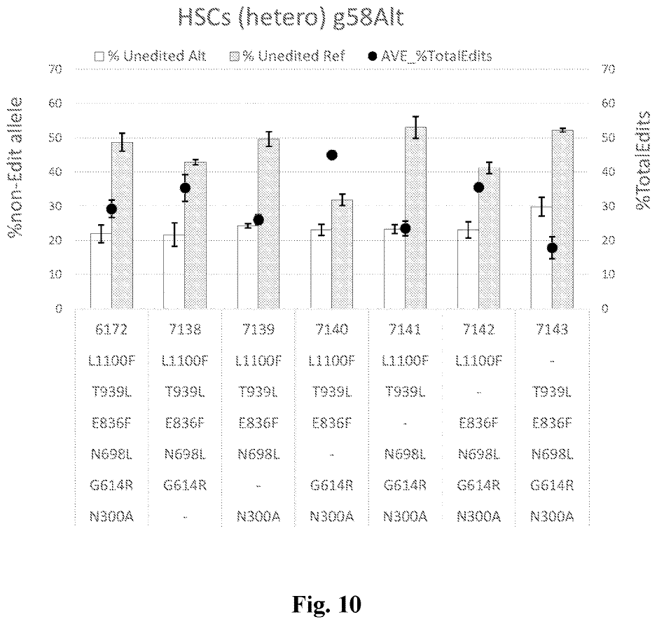

Since V6172 has a higher level of specificity, as manifested in its ability to discriminate between alleles based on a SNP, we further tested the contribution of removing single mutations from V6172 in heterozygote HSCs to g58 using the more challenging Alt guide version (FIG. 10). The same trend was seen for discrimination as for fidelity on g58Alt, where the discrimination decreases upon removing N300A (V7138), N698L (V7140), and T939L (V7142).

Designing Variant with Improved Activity and Fidelity Based on Variant 6172

Variant V7765 (which has the following substitutions relative to WT OMNI-50: N300A, G614R, N698L, S779P, E836F, T939L, L1100F, S1339R) is a rationally designed variant based on variant V6172 and has increased activity and specificity. Two additional mutations that were identified during screening were introduced: S779P and S1339R, which had proved to increase activity in V6552 (Table 2).

### S779P Substitution Increased OMNI-50 Thermal Stability

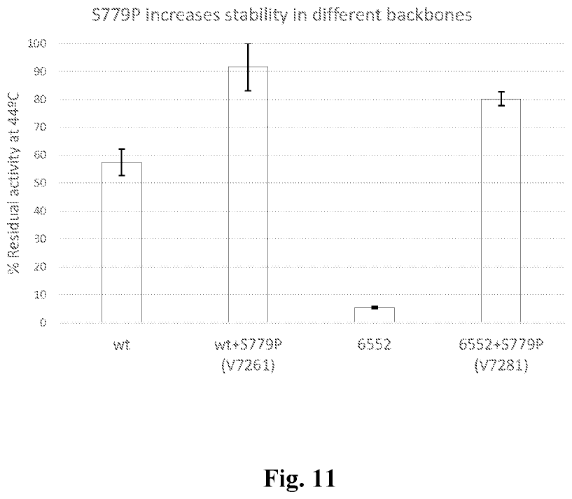

Variants of WT OMNI-50 were screened, and it was identified that a single substitution at position S779 in WT OMNI-50 to a proline residue (i.e. S779P, which forms variant V7261) significantly increased the thermal stability of the protein as measured by an increase in the residual activity at 44° C. (FIG. 11). The same effect was also observed when S779P was introduced to variant V6552 (i.e. variant V7281, see Table 2). These results indicate that S779P is a global stabilizing mutation that increases protein thermal stability on different mutational backbones.

### Increased Activity of OMNI-50 Variants V6552, V6172, V7765, and Increased Fidelity of V7765 on Different Targets

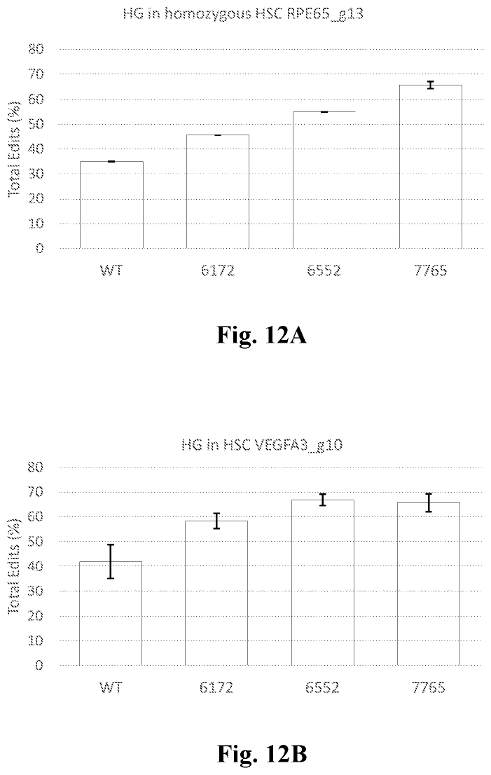

OMNI-50 variant V6552, OMNI-50 variant V6172, and OMNI-50 variant V7765 were designed to have higher activity than WT OMNI-50. To characterize their activity, we expressed and purified them in HG format and tested their activity as RNPs in HSC cells homozygous for the target sites RPE65 and VEGFA3 with their corresponding guide sequences (Table 3, FIGS. 12A-12B). Indeed, V6552, V6172, and V7765 show higher activity at both targets, where V7765 shows the highest activity.

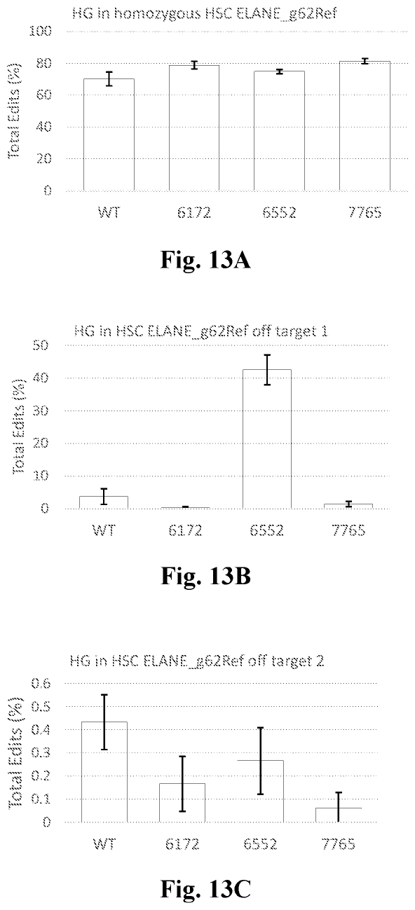

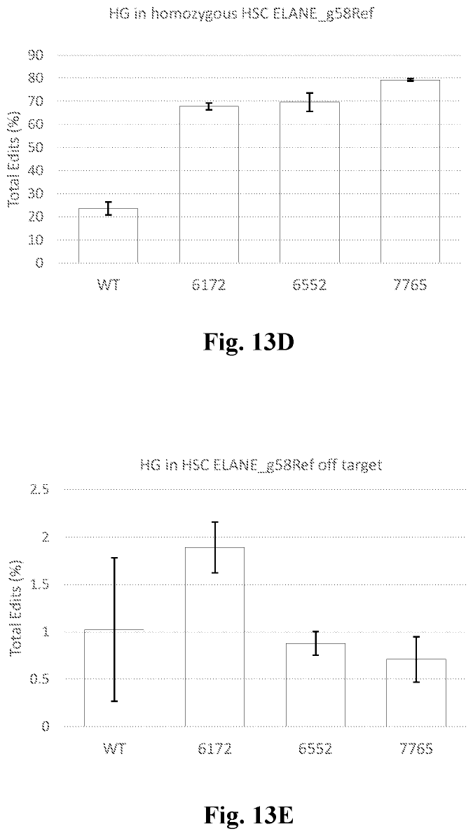

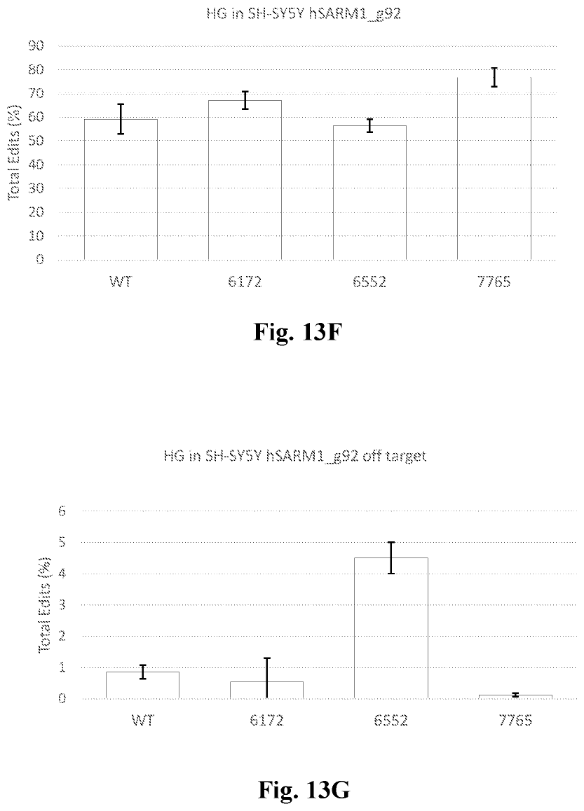

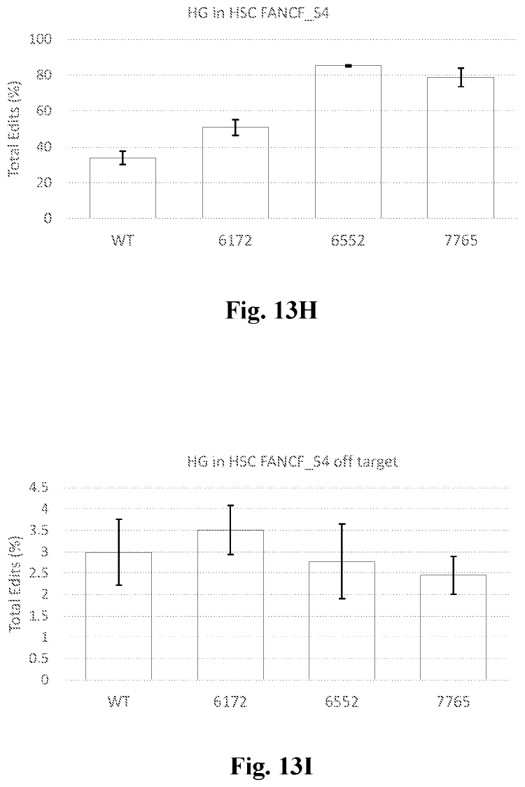

These variants were then tested on targets with known off-target sites in order to characterize their activity and specificity. Using target ELANE_g62Ref, all variants have high activity, however, V6172 and V7765 show higher fidelity for both off-target sites (FIGS. 13A-13C). For ELANE_g58Ref, variant V7765 shows the highest activity and fidelity (FIGS. 13D-13E). The variants were tested for target hSARM1_g92 in SH-SY5Y cells, and there as well V7765 shows the highest activity and fidelity (FIGS. 13F-13G). In FANCF it was shown that V7765 has higher activity than WT OMNI-50 and similar activity on the off-target (FIGS. 13H-13I).

TABLE 5

| | |
|---|---|
| ELANE Target 58 site sequence (ref) (PAM in | CAGCTGCGGGAAAGGGATTCCCAGG (SEQ ID NO: 107) |
| bold) | |
| ELANE Target 58 site sequence (Alt) (PAM in | CAGCTGCGGGAATGGGATTCCCAGG (SEQ ID NO: 108) |
| bold, SNP underlined) | |
| ELANE Target 58 (Ref) Off-target sequence | CAGCTGAGGGAAAGGAATTCCCAGG (SEQ ID NO: 109) |
| (PAM in bold) | |
| ELANE Target 62 site sequence (Ref) (PAM in | GTGTCAAGCCCCAGAGGCCACAGGG (SEQ ID NO: 110) |
| bold, SNP underlined) | |
| ELANE Target 62 site sequence (Alt) (PAM in | GTGTCAAGCCCCAGAGGACACAGGG (SEQ ID NO: 111) |
| bold, SNP underlined) | |
| ELANE Target 62 (Ref) Off-target sequence | AAGCCAAACCCCAAAGGCCACACGG (SEQ ID NO: 112) |
| (PAM in bold) | |
| hRPE65 Target g13 site sequence (PAM in bold) | AGAAGTCCTAATGGTAGCACCTGGG (SEQ ID NO: 143) |
| hSARM1 Target g92 site sequence (PAM in | CCTGTACTGGTGGCAAACCCAGTGG (SEQ ID NO: 144) |
| bold) | |
| hSARM1 Target g92 off site sequence (PAM in | GCAGTACAGGTGGCAAACCCAGAGG (SEQ ID NO: 145) |
| bold) | |
| FANCF Target s4 site sequence (PAM in bold) | GTGCTGCAGAAGGGATTCCATGAGG (SEQ ID NO: 146) |
| FANCF Off-Target s4 site sequence (PAM in | ATGCTGCAGAAGGGATTCCAAGGGG (SEQ ID NO: 147) |
| bold) | |
| VEGFA3 Target g10 sequence (PAM in bold) | TGGGTGAGTGAGTGTGTGCGTGTGG (SEQ ID NO: 148) |
| ELANE Target 38 site sequence (PAM in bold) | TCACAGCGGGTGTAGACTCCGAGGG (SEQ ID NO: 113) |
| ELANE Target 35 site sequence (PAM in bold) | GCAGTCCGGGCTGGGAGCGGGTGGG (SEQ ID NO: 114) |
| ELANE Target 35 Off-target sequence (PAM in | ACAGTCCTGGCTGGGAGCAGGTGGG (SEQ ID NO: 115) |
| bold) | |
| hSARMI Target 13 site sequence (PAM in bold) | TGCTCAGACACGCGGTGCAGCAGGG (SEQ ID NO: 116) |
| hSARMI Target g68 site sequence (PAM in | GTTTGCCACCAGTACAGGGCAGCGG (SEQ ID NO: 117) |
| bold) | |
| hRPE65 Target g13 site sequence (PAM in bold) | AGAAGTCCTAATGGTAGCACCTGGG (SEQ ID NO: 118) |
| OMNI-50 Guide RNA molecule 22-nucleotides | CAGCUGCGGGAAAGGGAUUCCC (SEQ ID NO: 119) |
| ELANE Target 58 ref spacer sequence (g58Ref) | |
| OMNI-50 Guide RNA molecule 22-nucleotides | CAGCUGCGGGAAUGGGAUUCCC (SEQ ID NO: 120) |
| ELANE Target 58 alt spacer sequence (g58Alt) | |
| OMNI-50 Guide RNA molecule 22-nucleotides | GUGUCAAGCCCCAGAGGCCACA (SEQ ID NO: 121) |
| ELANE Target 62 ref spacer sequence (g62Ref) | |
| OMNI-50 Guide RNA molecule 22-nucleotides | GUGUCAAGCCCCAGAGGACACA (SEQ ID NO: 122) |
| ELANE Target 62 alt spacer sequence (g62Alt) | |
| OMNI-50 Guide RNA molecule 22-nucleotides | UCACAGCGGGUGUAGACUCCGA (SEQ ID NO: 123) |
| ELANE Target 38 ref spacer sequence (g38Ref) | |
| OMNI-50 Guide RNA molecule 22-nucleotides | GCAGUCCGGGCUGGGAGCGGGU (SEQ ID NO: 124) |
| ELANE Target 35 ref spacer sequence (g35Ref) | |
| OMNI-50 Guide RNA molecule 22-nucleotides | UGCUCAGACACGCGGUGCAGCA (SEQ ID NO: 125) |
| hSARMI Target 68 spacer sequence (g68) | |
| OMNI-50 Guide RNA molecule 22-nucleotides | GUUUGCCACCAGUACAGGGCAG (SEQ ID NO: 126) |
| hSARMI Target 13 spacer sequence (g13) | |
| OMNI-50 Guide RNA molecule 22-nucleotides | AGAAGUCCUAAUGGUAGCACCU (SEQ ID NO: 127) |
| hRPE Target 65 spacer sequence (g65) | |
| OMNI-50 Guide RNA molecule scaffold | GUUUGAGAGUUAUGAAAAUGACGAGUUCAAAUAAAA |
| sequence | AUUUAUUCAAACCGCCUAUUUAUAGGCCGCAGAUGU |
| | UCUGCUUU (SEQ ID NO: 128) |
| OMNI-50 Guide RNA molecule 22-nucleotides | AGAAGUCCUAAUGGUAGCACCU (SEQ ID NO: 149) |
| hRPE65 Target 13 spacer sequence (g13) | |
| OMNI-50 Guide RNA molecule 22-nucleotides | CCUGUACUGGUGGCAAACCCAG (SEQ ID NO: 150) |
| hSARMI Target g92 spacer sequence (g92) | |
| OMNI-50 Guide RNA molecule 22-nucleotides | GCAGUACAGGUGGCAAACCCAG (SEQ ID NO: 151) |
| hSARMI Target g92 off site spacer sequence | |
| (g92_OT) | |
| OMNI-50 Guide RNA molecule 22-nucleotides | GUGCUGCAGAAGGGAUUCCAUG (SEQ ID NO: 152) |
| FANCF Target s4 site spacer sequence (g4) | |
| OMNI-50 Guide RNA molecule 22-nucleotides | AUGCUGCAGAAGGGAUUCCAAG (SEQ ID NO: 153) |
| FANCF off Target s4 site spacer sequence | |
| (g4_OT) | |
| OMNI-50 Guide RNA molecule 22-nucleotides | UGGGUGAGUGAGUGUGUGCGUG (SEQ ID NO: 154) |
| VEGFA3 Target g10 spacer sequence (g10) | |

1. A composition comprising a non-naturally occurring nuclease variant having at least 90% identity to SEQ ID NO: 1 and comprising an amino acid substitution in at least one of the following positions: N300, G614, N698, E836, T939, L1100, D252, D281, L302, N368, S1339, and S779 relative to SEQ ID NO: 1.

2. The composition of claim 1, wherein the nuclease variant comprises an amino acid substitution in at least one of the following positions: L1100, S1339, and/or S779.

3. The composition of claim 1, wherein the nuclease variant comprises an amino acid substitution at L1100.

4. The composition of claim 1, wherein the nuclease variant comprises an amino acid substitution at S1339.

5. The composition of claim 1, wherein the nuclease variant comprises an amino acid substitution at positions L1100 and S1339.

6. (canceled)

7. (canceled)

8. The composition of claim 1, wherein the nuclease variant comprises at least one of the following:
an amino acid substitution at position L1100, wherein the amino acid substituted for leucine is a histidine (L1100H), phenylalanine (L1100F), tryptophan (L1100W), or tyrosine (L1100Y);
an amino acid substitution at position S1339, wherein the amino acid substituted for serine is an arginine (S1339R), lysine (S1339K), or histidine (S1339H); and/or
an amino acid substitution at position S779, wherein the amino acid substituted for serine is a glycine (S779G), alanine (S779A), valine (S779V), cysteine (S779C), proline (S779P), leucine (S779L), isoleucine (S779I), methionine (S779M), tryptophan (S779W), phenylalanine (S779F), aspartic acid (S779D), asparagine (S779N), or histidine (S779H).

9. The composition of claim 1, wherein the nuclease variant comprises at least one of the following amino acid substitutions: L1100F, S1339R, and/or S779P.

10. (canceled)

11. (canceled)

12. (canceled)

13. (canceled)

14. The composition of claim 1, wherein the nuclease variant comprises an amino acid substitution in at least one of the following positions: N300, G614, N698, E836, and/or T939.

15. The composition of claim 1, wherein the nuclease variant comprises at least one of the following amino acid substitutions: N300A, G614R, N698L, E836F, and/or T939L.

16. The composition of claim 1, wherein the nuclease variant comprises an amino acid substitution in at least one of the following positions: D252, D281, L302, and/or N368.

17. The composition of claim 1, wherein the nuclease variant comprises at least one of the following amino acid substitutions: D252Y, D281V, L302N, and/or N368S.

18. (canceled)

19. (canceled)

20. The composition of claim 1, wherein the nuclease variant has at least 97% identity to SEQ ID NO: 1.

21. The composition of claim 1, wherein the nuclease variant comprises at least one of the following amino acid substitutions: N300A, G614R, N698L, E836F, T939L, L1100F, D252Y, D281V, L302N, N368S, S1339R, and S779P.

22. The composition of claim 1, wherein the nuclease variant comprises an amino acid sequence of any one of SEQ ID NOs: 2-30.

23. The composition of claim 1, wherein the nuclease variant further comprises at least one nuclear localization sequence (NLS).

24. (canceled)

25. (canceled)

26. (canceled)

27. (canceled)

28. The composition of claim 1, further comprising a single-guide RNA (sgRNA) molecule, crRNA molecule, and/or a tracrRNA molecule, or a DNA molecule encoding a single-guide RNA (sgRNA) molecule, crRNA molecule, and/or a tracrRNA molecule.

29. A method of binding and/or modifying a DNA target site in a cell or cell-free system, the method comprising delivering to the cell or cell-free system the composition of claim 1.

30. The method of claim 29, wherein the binding and/or modifying occurs in a eukaryotic cell or prokaryotic cell.

31. (canceled)

32. (canceled)

33. (canceled)

34. The method of claim 29, wherein the DNA target site is located in a gene selected from the group consisting of ELANE, CXCR4, EMX, RyR2, KNCQ1, KCNH2, SCN5a, GBA1, GBA2, Rhodopsin, GUCY2D, IMPDH1, FGA, BEST1, PRPH2, KRT5, KRT14, ApoA1, STAT3, STAT1, ADA2, RPS19, SBDS, GATA2, RPE65, LDLR, ANGPTL3, B2M, TRAC, TCF4, TGFBi, PAX6, C3, LRRK2, SARM1, SAMD9, SAMD9L, HAVCR2, CD3E, APLP2, CISH, TIGIT, TNNT2, TNN, MYH7, VEGFA3, FANCF, and HLA-E.

35. (canceled)

36. (canceled)

37. (canceled)

38. A modified cell obtained by the method of claim 29.

39. The modified cell of claim 38, wherein the cell is capable of engraftment.

40. The modified cell of claim 38, wherein the cell is capable of giving rise to progeny cells after engraftment.

41. (canceled)

42. (canceled)

43. The modified cell of claim 38, wherein the cell is selected from the group consisting of a hematopoietic stem cell, a progenitor cell, a CD34+ hematopoietic stem cell, a bone marrow cell, and a peripheral mononucleated cell.

44. A composition comprising a modified cell of claim 38 and a pharmaceutically acceptable carrier.

45. (canceled)

46. (canceled)

47. The method of claim 29, wherein the DNA target site is located in a gene selected from the group consisting of ELANE, RPE65, SARM1, VEGFA3, and FANCF.
